SEQUENCE LISTING (2 sequences)

SEQ ID NO: 1 - protein, 96 aa, synthetic construct
MEQAPEDQGPQREPHNEWTLELLEELKNEAVRHFPRIWLHGLGQHIYETYGDTWAGVEAIIRILQQLLFIHFRIGCRHSRIGVTQQRRARNGASRS

SEQ ID NO: 2 - protein, 78 aa, synthetic construct
MEQAPEDQGPQREPHNEWTLELLEELKNEAVRHFPRIWLHGLGQHIYETYGDTRAGVEAIIRILRQLLFIHFRIGCQH

(19) United States
(12) Patent Application Publication
(10) Pub. No.: US 2024/0293466 A1
(43) Pub. Date: Sep. 5, 2024

(54) DIFFERENTIAL CXCR4 EXPRESSION ON HEMATOPOIETIC PROGENITOR CELLS VERSUS STEM CELLS DIRECTS HOMING AND LONG-TERM ENGRAFTMENT

(71) Applicant: CHILDREN'S HOSPITAL MEDICAL CENTER, Cincinnati, OH (US)

(72) Inventor: Punam MALIK, Cincinnati, OH (US)

(21) Appl. No.: 18/578,381

(22) PCT Filed: Jul. 12, 2022
(86) PCT No.: PCT/US2022/073641

§ 371 Date: Jan. 11, 2024

Related U.S. Application Data
(60) Provisional application No. 63/220,792, filed on Jul. 12, 2021.

(51) Int. Cl. A61K 35/28; A61K 48/00; C07K 14/005; C07K 14/715; C12N 5/0789; C12N 15/86

(52) U.S. Cl. CPC A61K 35/28; A61K 48/0025; C07K 14/005; C07K 14/7158; C12N 5/0647; C12N 15/86; C07K 2319/50; C12N 2510/00; C12N 2740/16022; C12N 2740/16023; C12N 2740/16042; C12N 2740/16043

(57) ABSTRACT

Compositions and methods for delivering a protein of interest such as a cell homing molecule (e.g., CXCR4) into host cells (e.g., to hematopoietic cells). The compositions and methods provided herein may be used to enhance homing and long-term engraftment of hematopoietic cells post transplantation.

### CROSS REFERENCE TO RELATED APPLICATIONS

This application claims the benefit of the filing dates of U.S. Provisional Application No. 63/220,792, filed Jul. 12, 2021, the entire contents of which are incorporated by reference herein.

### SEQUENCE LISTING

The instant application contains a Sequence Listing which has been filed electronically in XML format and is hereby incorporated by reference in its entirety. Said XML copy, created on Jul. 12, 2022, is named 112149-0249-70048WO00.xml and is 2,840 bytes in size.

### BACKGROUND OF THE INVENTION

Transplantation of hematopoietic stem cells (HSC) is a lifesaving procedure used to treat a variety of blood disorders and cancers. Gene therapy is a variant of autologous HSC transplantation, in which a person's own HSC are re-infused following gene correction, avoiding the risk of graft-vs-host disease or the limitations of a matched donor. The success of genetic therapies is dependent upon the ability to achieve sufficient levels of engraftment of genetically modified (GM) HSC by maintaining their self-renewal and differentiation capacity, while also achieving high levels of gene transfer.

Homing and engraftment of sufficient GM HSC are significant rate-limiting steps for the success of gene therapy. Accordingly, it is of great interest to develop methods to enhance homing and engraftment of HSCs, specifically genetically modified HSCs.

### SUMMARY OF THE INVENTION

The present disclosure is based, at least in part, on the discovery that a high level of CXCR4 expression on hematopoietic progenitor cells (HPCs) confers a homing and engraftment advantage to the HPCs over hematopoietic stem cells (HSCs), which express a lower level of CXCR4. An HSC-enriched cell population (with CXCR4^{high} HPCs removed) showed improved long-term homing and engraftment following direct bone marrow transplantation. The present disclosure is also based, at least in part, on the development of a system for transient CXCR4 expression (via protein delivery) in hematopoietic cells, thereby enhancing homing and engraftment (e.g., long-term engraftment) of the modified hematopoietic cells. This method of protein delivery can also be used to deliver specific homing molecules or other proteins to a variety of other cell types (e.g., to improve homing of lymphocytes, NK cells).

Accordingly, provided herein are compositions and methods for enhancing hematopoietic stem cell (e.g., genetically modified HSCs) homing and/or engraftment (e.g., long-term engraftment) post transplantation, e.g., using the modified hematopoietic cells disclosed herein or HSC-enriched cell population via direct bone marrow transplantation. Also provided herein are systems and methods for delivering a cell homing molecule such as CXCR4, optionally together with a transgene of interest, to hematopoietic cells, and the modified hematopoietic cells thus produced, which have improved homing and engraftment capacities.

Accordingly, some aspects of the present disclosure feature a virus-like particle (VLP), comprising a protein shell, which comprises a protein of interest such as a cell homing molecule and one or more viral surface proteins. When entering a host cell, the protein of interest (e.g., the cell homing molecule) detaches from the VLP and delivers to the host cell. In some instances, the protein of interest such as the cell homing molecule may be displayed on the surface of the host cell. In some embodiments, the VLP may further comprise a nucleic acid, which is encapsulated by the protein shell. The nucleic acid may comprise a transgene of interest, which may be co-delivered to a cell with the protein of interest (e.g., the cell homing molecule). In some examples, the VLP is a lentivirus-like particle.

In some embodiments, the cell homing molecule can be a fusion protein comprising a CXCR4 polypeptide fused to a viral protein R (Vpr) fragment, which may be devoid of toxicity. In some examples, the Vpr fragment comprises a truncation of a carboxyl terminal region (e.g., residues 79-96 of a wild-type Vpr) and/or one or more mutations at positions W54, Q65, and R77 as compared with the wild-type counterpart. In some instances, the one or more mutations are amino acid substitutions of W54R, Q65R, and R77Q. Alternatively or in addition, the Vpr fragment comprises an amino acid sequence at least 80% identical to residues 1-78 of SEQ ID NO:1 and comprises one or more of the mutations. In one specific example, the Vpr comprises the amino acid sequence of SEQ ID NO:2.

In some embodiments, the fusion protein may further comprise a protease cleavage peptide, which can be located between the CXCR4 polypeptide and the Vpr fragment. Upon cleavage at the protease cleavage peptide via a suitable protease (which may be packed into the VLP), the CXCR4 polypeptide can be released from the fusion protein. When the VLP is a lentiviral particle, the protease cleavage peptide may comprise a polybasic cleavage site (PCS).

Any of the CXCR4-Vpr fusion proteins disclosed herein, as well as nucleic acids encoding such, is also within the scope of the present disclosure. The nucleic acid encoding such a fusion protein may be a vector, for example, an expression vector (e.g., a viral vector such as a lentiviral vector), which comprises a nucleotide sequence encoding the fusion protein in operable linkage to a suitable promoter. Also provided herein are host cells comprising any of the nucleic acids encoding the fusion protein.

In other aspects, the present disclosure provides a method for delivering a cell homing molecule with or without a transgene to cells. Such a method may comprise: contacting a VLP as disclosed herein with a population of cells to allow entry of the VLP into the cells, thereby delivering the cell homing molecule, and optionally the transgene, to the cells.

In some embodiments, the population of cells comprise hematopoietic cells. In other embodiments, the hematopoietic cells are umbilical cord blood (UCB) cells. In yet other embodiments, the hematopoietic cells comprise immune cells, for example, T cells, NK cells, or a combination thereof. A transgene encoding a chimeric antigen receptor may be delivered to the immune cells, together with the cell homing molecule.

In some embodiments, the hematopoietic cells are hematopoietic progenitor cells (HPCs), hematopoietic stem cells (HSCs), or a combination thereof. For example, the hematopoietic cells may comprise CD34+ cells. In other examples, the hematopoietic cells may comprise CD34⁺ and CD38⁺ cells (CD34⁺/CD38⁺ cells). Alternatively, the hematopoietic cells may comprise CD34⁺ and CD38⁻ cells (CD34⁺/CD38⁻ cells). In yet other examples, the hematopoietic cells comprise CD34+, CD38−, and CD90+ cells (CD34⁺/CD38⁻/CD90⁺ cells). In some examples, the modified hematopoietic cells may be CD34⁺ and comprise one or more of the following features: CD38⁻, CD90⁺, CD45RA⁻, CD49F⁺, and CD133⁺.

In addition, the present disclosure provides a population of hematopoietic cells comprising modified hematopoietic cells, which comprises an exogenous cell homing molecule and optionally a transgene of interest. In some embodiments, the cell homing molecule can be a CXCR4 protein, for example, the CXCR4-Vpr fusion protein disclosed herein. In some embodiments, the modified hematopoietic cells comprise CD34⁺ cells. In other embodiments, the modified hematopoietic cells comprise CD34⁺ and CD38⁺ cells. Alternatively, the modified hematopoietic cells comprise CD34⁺ and CD38⁻ cells. In yet other embodiments, the modified hematopoietic cells comprise CD34⁺, CD38⁻, and CD90⁺ cells. In some examples, the modified hematopoietic cells may be CD34⁺ and comprise one or more of the following features: CD38⁻, CD90⁺, CD45RA⁻, CD49F⁺, and CD133⁺. In some instances, the population of hematopoietic cells may be produced by any of the preparation methods disclosed herein.

In yet other aspects, the present disclosure provides a method for delivering any of the modified hematopoietic cells disclosed herein to a subject. The method comprises administering a population of hematopoietic cells comprising the modified hematopoietic cells to a subject in need thereof. The modified hematopoietic cells have enhanced homing and/or engraftment capacity as compared with counterpart hematopoietic cells lacking the exogenous cell homing molecule, e.g., the CXCR4 protein. In some instances, the subject is a human patient in need of stem cell transplantation and/or gene therapy.

In some embodiments, the population of hematopoietic cells can be administered to the subject by intravenous injection. Alternatively, the population of the hematopoietic cells can be administered to the subject by intra-bone marrow (IBM) injection.

Further, the present disclosure features a method for improving engraftment of long-term repopulating cells (LTRCs) in a subject. The method comprises administering a population of hematopoietic cells comprising LTRCs to a subject in need thereof by intra-bone marrow (IBM) injection. The LTRCs comprise CD34⁺ and CD38⁻ cells, and the population of hematopoietic cells comprises no more than 10% CD34⁺ and CD38⁺ cells. In some embodiments, the LTRCs comprise CD34⁺, CD38⁻, and CD90⁺ cells. In some embodiments, LTRCs are CD34⁺/CD38⁻/CD90⁺/CD45RA⁻ cells. In some embodiments, the LTRCs are genetically modified to carry a transgene of interest. In some embodiments, the subject is a human patient in need of stem cell transplantation and/or gene therapy.

In any of the methods disclosed herein, the hematopoietic cells may be autologous to the subject receiving such. Alternatively, the hematopoietic cells may be allogeneic to the subject receiving such.

In addition, the present disclosure features a gene therapy method, comprising:
(i) isolating a population of hematopoietic cells from a first subject;
(ii) introducing into the population of hematopoietic cells (a) a transgene encoding a therapeutic agent and (b) a cell homing molecule to produce a population of modified hematopoietic cells; and
(iii) administering the modified hematopoietic cells to a second subject.

In some embodiments, the first subject and the second subject are an identical human patient (autologous therapy). In other embodiments, the first subject and the second subject are different human patients (allogeneic therapy). In some embodiments, the transgene and the cell homing molecule are introduced into the population of hematopoietic cells concurrently in step (ii). In some examples, the cell homing molecule is a CXCR4 protein, e.g., any of the CXCR4-Vpr fusion proteins disclosed herein.

In some embodiments, the population of hematopoietic cells isolated in step (i) may comprise CD34+ cells. In some embodiments, the population of hematopoietic cells isolated in step (i) may comprise CD34⁺ and CD38⁺ cells. In some embodiments, the population of hematopoietic cells isolated in step (i) may comprise CD34⁺ and CD38⁻ cells. In some embodiments, the population of hematopoietic cells isolated in step (i) may comprise CD34⁺, CD38⁻, and CD90⁺ cells.

In some embodiments, the modified hematopoietic cells can be administered to the second subject by intravenous infusion. In other embodiments, the modified hematopoietic cells can be administered to the second subject by intra-bone marrow injection.

Also within the scope of the present disclosure are any of the hematopoietic cell populations (e.g., modified or HSC-enriched) for use in treating a target disease (e.g., those disclosed herein), as well as uses of such hematopoietic cell populations for manufacturing a medicament for use in treatment of the target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1H include diagrams showing that hematopoietic progenitor cells (HPC) have a preferential engraftment advantage when transplanted in the bone marrow directly in NSG Mice. FIGS. 1A-1D: Irradiated NSG mice were transplanted with MPB CD34+HSPC that were transduced with a GFP-encoding LV vector (gene transfer 63-66%) either IV or IBM at the indicated cell doses. Human cell engraftment was analyzed by determining the percentage of human CD45+ (hCD45+) cells and GM human CD45+ (hCD45+GFP+) cells in the bone marrow obtained via bone marrow aspirate at 12 weeks to assess short-term engraftment (FIGS. 1A-1B) and from pooled bone marrow from femurs, tibias, and iliac crests at 24 weeks to determine long-term human engraftment (FIGS. 1C-1D). Bars represent median engraftment. Each symbol represents an individual mouse; N=18-20 mice per cell dose per transplant method; mice were transplanted using 4 unique MPB donors in 4 experiments; statistical analysis was performed using t-tests between the IV and IBM groups at each cell dose. FIGS. 1E-1H: Lineage output of the human xenograft. Bone marrow was stained antibodies specific for T cell lineage (hCD3), B cell lineage (hCD19), myeloid lineage (hCD33) and HSPC (hCD34) to determine the lineage output of the transplanted total HSPC (FIGS. 1E and 1G) and GM HSPC (FIGS. 1F and 1H) at 12 weeks and 24 weeks post-transplant. *p<0.05, **p<0.01

FIGS. 2A-2G include diagrams showing the homing of IV-and IBM-transplanted HSPC and expression of homing receptors on HSC and HPC. FIG. 2A: MPB CD34+HSPC (3×10⁶ per mouse) were injected IV, IBM in 10 uL volume using a standard 0.5 ml syringe, or slow IBM using a Hamilton syringe to deliver the cells over 1 minute and homing of CD34+ cells to the bone marrow analyzed 20-22 hours later. To determine if mechanical pressure in the injected femur alters homing, irradiated CD34− cells were injected IBM and CD34+ cells were injected IV into the same animal. Homing was determined in the injected femurs (IF) and non-injected femurs (nonIF) with IBM injections, and in both femurs (BF) combined with IV injection. Symbols represent individual animals; N=3 mice per group; statistical analysis was performed using 2-way ANOVA. FIGS. 2B-2C: For IBM injections, bone marrow from the IF and nonIF was analyzed for expression of CXCL12 and VCAM-1 on mouse stromal cells (hCD45−, PKH26−, mouse lineage-mCD45−mCD51+ cells). N=5 mice per group; data was normalized to IV injected group; statistical analysis was performed by Mann-Whitney test. FIGS. 2D-2E: CXCR4 and VLA-4 homing receptor expression of CD34+HSPC, CD34+CD38+HPC, and CD34+CD38-CD90+HSC-enriched populations. Data was normalized to CXCR4 and VLA-4 expression on CD34+HSPC. Symbols represent unique MPB donors; N=5 per group; statistical analysis was performed by Mann-Whitney test. FIG. 2F: Adhesion of CD34+HSPC, CD34+CD38+HPC and CD34+CD38−CD90+HSC to CXCL12. Symbols represent unique MPB donors; N=5 per group; statistical analysis was performed by Mann-Whitney test. FIG. 2G: Binding of CD34+HSPC, CD34+CD38+HPC, and CD34+CD38−CD90+HSC to the VLA-4 affinity detection ligand, LDV-FITC. Data was normalized to LDV-FITC expression in CD34+HSPC. Symbols represent unique MPB donors; N=3 per group; statistical analysis was performed by Mann-Whitney test. *p<0.05, **p<0.01. In FIGS. 2B-2E and 2G, flow cytometry data was normalized to the indicated group to account for variability in MFI between different experiments/donors.

FIGS. 3A-3F include diagrams showing that the level of expression of CXCR4, not VLA-4 on HSPC directs homing into bone marrow with IBM transplants. FIGS. 3A-3C: High CXCR4 expression on HPC mediates their preferential homing locally with IBM transplant: CD34+HSPC were transduced with a BFP LV vector, and the CD34+CD38+BFP+HPC were sorted by flow cytometry 72 hours post gene transfer. HPC were blocked with AMD3100 (a CXCR4 antagonist) or BIO5192 (a VLA-4 antagonist) before IV or IBM delivery (FIG. 3A). Homing of hCD34+cells was analyzed in the injected femur (IF) and non-injected femur (nonIF) for IBM injected mice, and in both femurs combined in IV injected mice. Data was normalized to IV injected control CD34+CD38+ cells and the fold increase is indicated within the bar. (FIGS. 3B-3C). N=5-6 mice per group; statistical analysis was performed by comparing the modes of delivery by Mann-Whitney test. FIGS. 3D-3F: Induction of high CXCR4 expression on CD34+CD38−HSC enriched population via gene transfer confers them with a homing advantage despite higher abundance of CD34+CD38+HPC. CD34+HSPC were transduced with a BFP LV vector or a BFP-CXCR4 LV vector and sorted for transduced CD34+CD38−BFP+cells and untransduced CD34+CD38+BFP−HPC at 72 hours. Transduced CD34+CD38−cells were mixed with untransduced CD34+CD38+cells and transplanted IV or IBM and homing of CD34+CD38−BFP+cells into bone marrow analyzed at 20-22 hours. FIG. 3E: CXCR4 expression on injected cells at time of transplant is shown and the MFI on injected cells at time of transplant was as follows: CD34+CD38−control 16,930, CD34+CD38+control 35,098, CD34+CD38−CXCR4 transduced 37,453. FIG. 3F: Homing in bone marrow is shown. N=3-10 mice per experimental arm; statistical analysis was performed by comparing the modes of delivery by Mann-Whitney test. *p<0.05, **p<0.01, ***p<0.001, ****p<0.0001.

FIGS. 4A-4D include diagrams showing that IBM transplant of an HSC-enriched population removes the competition from HPC and enhances long-term repopulation compared to IV delivery. CD34+CD38−HSC-enriched cells were obtained using immunomagnetic sorting, transduced with a GFP LV vector (gene transfer efficiency was 69%) and transplanted into NSG mice IV or IBM in two limiting dilution doses. FIGS. 4A-4B: Long-term (24 week) engraftment of total (hCD34+) and GM human (hCD45+GFP+) cells is shown. Symbols represent individual animals; N=3-4 mice per treatment condition; statistical analysis was performed by comparing the modes of delivery at the different cell doses by Mann-Whitney test. FIGS. 4C-4D: Lineage output at 24 weeks shows that the long-term human graft was multi-lineage, composed of B, T, myeloid cells and CD34+HSPC. *p<0.05

FIGS. 5A-5G include diagrams showing that transiently increased expression of CXCR4 on GM CD34+38−cells via protein delivery in a LV particle significantly increases their homing and long-term engraftment. FIGS. 5A-5B: A GFP-encoding LV vector was packaged either using standard packaging (LV) alone or Vpr^{MT}-CXCR4 plasmid in addition, to package the CXCR protein attached to the LV vector capsid (LV^{CXCR4}). CXCR expression on MPB CD34+cells transduced with LV^{CXCR4} vector compared to cells transduced with the control LV vector 24 hours following gene transfer is shown. FIG. 5C: The time course of CXCR4 expression in LV^{CXCR4} HSPC, normalized to that of control LV HSPC is shown, with expression peaking at 24 hours that returns to baseline expression by 72 hours. Symbols represent individual MPB donors; N=3; statistical analysis was performed by Mann-Whitney test comparing the IV and IBM groups. FIG. 5D: Experimental schema for assessment of homing and engraftment of CD34+CD38−cells transduced with GFP LV or GFP LV^{CXCR4} that were transplanted into NSG mice either via IV or IBM delivery. The number of animals used for the homing experiment and engraftment experiments is indicated under each experimental arm. FIG. 5E: Homing of CD34+CD38−cells in the bone marrow. ^{\hat{ }} indicates injected femur (IF), ^{#} indicates non-injected femur (nonIF), and ^{\hat{ }#} indicates both femurs (BF). Data was normalized to IV injected control CD34+CD38−cells and the fold increase is indicated within the bar. Symbols represent individual mice; statistical analysis performed by Mann-Whitney tests. FIGS. 5F-5G: Long-term engraftment of CD34+CD38−cells was assessed by determining the percentage of human CD45+GFP-cells (FIG. 5F) and human CD45+GFP+(FIG. 5G) cells 24 weeks following transplant. Data was normalized to IV transplanted GFP LV transduced CD34+CD38−cells and the fold increase is indicated within the bar. Gene transfer was 60% with GFP LV and 52% with GFP LV^{CXCR4}. Symbols represent individual mice; statistical analysis was performed by comparing the modes of delivery by Mann-Whitney test. *p<0.05, **p<0.01

FIG. 6 is a schematic illustration depicting an exemplary NSG model to study engraftment of GM adult HSPC derived from G-CSF mobilized peripheral blood. The mice were transplanted limiting dilution CD34+cell doses after transduction of the cells with a GFP-encoding LV vector either IV or IBM. Indicated cell doses were injected after NSG mice received 280cGy irradiation. At 12 weeks after transplant, short-term engraftment was analyzed via bone marrow aspirate from one femur (the non-injected femur in IBM mice). At 24 weeks after transplant, mice were sacrificed and analyzed for long-term engraftment.

FIGS. 7A-7J include diagrams showing the effect of ex vivo culture, cell cycle, and transduction on homing receptor expression on HSPC. FIGS. 7A-7B: Cell surface expression of VLA-4 (FIG. 7A) and CXCR4 (FIG. 7B) on CD34+HSPC increases with increasing time in culture. Each symbol represents a unique MPB donor; N=3; statistical analysis was performed using ANOVA. FIGS. 7C-7D and 7F-7G: Cell cycle status of CD34+HSPC in culture and its effect on CXCR4 and VLA-4 homing receptor expression. FIGS. 7C and 7F: CD34+HSPC are largely quiescent when harvested but increasing numbers of HSPC enter cell cycle with increasing time in culture. Of the HSPC population, significantly higher numbers of the CD34+CD38+HPC are in cycle, as compared to relatively low numbers of cycling HSC (CD34+38−90+). N=3; statistical analysis was performed using ANOVA. FIGS. 7D and 7G: Both VLA-4 and CXCR4 expression is increased on cycling HSPC. Each symbol represents a unique MPB donor; N=3; statistical analysis was performed using ANOVA. FIGS. 7E and 7H: Homing receptor expression is cell cycle dependent. Non-cycling (G0 phase) CD34+38+HPC and CD34+38−HSC enriched cells expressed similar levels of homing receptor expression. FIGS. 7I-7J: Effect of transduction on homing receptor expression. CXCR4 expression in transduced and untransduced cells is similar, but VLA-4 expression is significantly higher on transduced HSPC. N=3 unique biological replicates; statistical analysis was performed by paired t tests between populations. *p<0.05, **p<0.01, ***p<0.001, ****p<0.0001

FIGS. 8A-8D include diagrams showing the development of CXCR4 protein delivery in the lentiviral (LV) vector. FIG. 8A: Construct Design. The amino acid (aa) sequence of Vpr and its mutant version generated is shown as SEQ ID NO: 1. Vpr, a small HIV accessory protein, is carried in viral particles bound to Gag, the capsid protein, via residues in its central region that folds into 3 α-helices. These are flanked by unstructured N-and C-terminal domains. The C-terminal domain imparts protein stability and has six arginine residues between positions 73 and 96 that potentiate nuclear localization, G2M arrest and apoptosis. S79 phosphorylation is important for cell cycle arrest. The VPR was truncated at the 78aa. Vpr^{R77Q} mutation was made in the third helix. The third helix forms a leucine-zipper like motif that interacts with DNA damage response (DDR) proteins via UNG2 via W54 and the ubiquitin-proteasome complex via DCAF via Q65. Vpr^{W54R} and Vpr^{Q65R} mutations were also made to abrogate binding to UNG2 and DCAF, preventing DDR, and eliminate all changes in the proteome normally triggered by Vpr upon viral entry, respectively. The triple mutated and truncated version of Vpr (Vpr^{MT}) (SEQ ID NO:2) was designed based on reported structure function analysis to abrogate its pathogenicity (residues associated with toxicity are highlighted in red) while retaining its structural features allowing Gag binding. Vpr^{MT} was fused to CXCR4 cDNA via the HIV-1 protease cleavage site (PCS) to generate Vpr^{MT}-CXCR4. FIG. 8B: Packaging of a lentiviral (LV) vector using the standard packaging plasmids Gag-Pol [gag forms the capsid protein, pol generates the reverse transcriptase (RT), integrase (IN) and protease (PR)], Rev (facilitates full length vector genome mRNA export), and envelope (VSV-G) and the transgene plasmid (that carries the marker/therapeutic transgene). FIG. 8C: LV vector packaged with the additional Vpr^{MT}-CXCR4 packaging plasmid (LV^{CXCR4}). FIG. 8D: LV^{CXCR4} vector-like particles (VLP) packaged without the vector genomic/transgene to generate empty LV particles.

FIGS. 9A-9H include diagrams showing that protein delivery of CXCR4 within a LV fused to Vpr^{MT} lacks the toxicity in CD34+HSPC that is normally associated with wild type Vpr protein in T lymphocytes. CD34+cells were either transduced with a GFP LV vector or a GFP LV^{CXCR4} vector. Gene transfer was comparable between groups for all donors. FIGS. 9A-9B: Cell cycle status was determined in CD34+HSPC and CD34+CD38−CD90+HSC populations 72 hours-post transduction. Earlier time-points did not have sufficient CD34+CD38−CD90+cells in G₂M phase for a meaningful comparison. FIGS. 9C-9D: Viable cell counts of CD34+HSPC and CD34+CD38−CD90+HSC were determined 48 hours post-transduction. Cell counts were normalized to untransduced (mock) control cell counts from each unique donor. Symbols represent unique MPB donors; N=3. FIGS. 9E-9F: Apoptosis was measured in CD34+HSPC and CD34+CD38−CD90+HSC populations 48 hours post-transduction via Annexin V staining. Symbols represent unique MPB donors; N=3. FIGS. 9G-9H: DNA damage response was assessed by γH2AX+cells in both CD34+ and CD34+CD38−CD90+HSC populations 48 hours post-transduction. Symbols represent unique MPB donors; N=3.

FIGS. 10A-10F include diagrams show that protein delivery of CXCR4 enhances long-term multi-lineage engraftment of CD34+CD38−HSC-enriched cells when transplanted IV or IBM. FIGs. A-D: Irradiated NSG mice were transplanted 0.25e5 (FIGS. 10A and 10C) or 0.5e5 (FIGS. 10B and 10D). CD34+CD38−HSC-enriched cells that were transduced with GFP LV or GFP LV^{CXCR4} (gene correction was 60% with GFP LV and 52% with GFP LVCXCR4) by either IV or via IBM injection at the indicated cell doses. Human cell engraftment was analyzed by determining the percentage of gene-modified/transduced human CD45+(hCD45+GFP+) (FIGS. 10A-10B) cells and the percentage of non-transduced (hCD45+GFP−) (FIGS. 10C-10D) cells in bone 24 weeks post-transplant. Symbols represent individual mice; N=3-5 mice per cell dose per group; statistical analysis was performed by comparing the modes of delivery by Mann-Whitney test. FIGS. 10E-10F: Lineage output of the human xenograft. Bone marrow was stained antibodies specific for T cell lineage (hCD3), B cell lineage (hCD19), myeloid lineage (hCD33) and HSPC (hCD34) to determine the lineage output of the transduced and untransduced HSC-enriched population 24 weeks post-transplant. *p<0.05, **p<0.01.

FIG. 11 includes a diagram showing that secondary transplants showed no difference in long term engraftment with IV or IBM delivery. Pooled bone marrow from femurs, tibias, and iliac crests from primary mice at 24 weeks was transplanted into sub-lethally irradiated secondary mice. Secondary mouse bone marrow was analyzed for human cell engraftment at 12 weeks. Bars represent median engraftment. Each symbol represents an individual mouse. N=9 secondary animals per group; Primary mice were transplanted from one MPB donor. Transduced cell engraftment is not shown as the overall percentage of transduced cells was very low and therefore not reliable. Symbols represent individual secondary mice; statistical analysis performed by Mann-Whitney test.

FIGS. 12A-12D include diagrams that show the analysis of blood and stromal cells following IV and IBM injection. FIG. 12A: Representative flow cytometry plots show gating for PKH26+(PKH+) cells from whole blood from mice bled at 6 hours post-transplantation after IV or IBM injection. FIG. 12B: Representative flow cytometry plots show gating for PKH26+(PKH+) cells from whole blood from mice bled at 22 hours post-transplantation after IV or IBM injection.

### DETAILED DESCRIPTION OF THE INVENTION

Transplantation of hematopoietic stem cells (HSCs), including genetically modified HSCs, has been used to treat a variety of blood disorders and cancers. Gene therapy (e.g., involving genetically modified HSCs) can be performed using viral vectors (e.g., lentiviral or γ-retroviral vectors). In general, gene therapy may involve: (1) harvest and enrichment of autologous CD34+hematopoietic stem and progenitor cells (HSPC), (2) ex vivo culture of the HSPCs for genetic manipulation, (3) chemotherapeutic conditioning of a patient (to open the bone marrow HSC niche), and (4) intravenous (IV) infusion of the genetically-modified (GM) HSPC. The rare HSC (1-2%) within the CD34⁺ HSPC population home and engraft the bone marrow HSC niche and result in long-term engraftment and GM multi-lineage blood cell progeny.

However, a substantial loss of hematopoietic stem and progenitor cells (HSPC) occurs from collection through transplant. As a result, a much higher HSPC dose is collected, genetically manipulated, and infused than is necessary for standard (non-gene therapy) autologous transplants. Obligate losses include those that occur during HSPC enrichment from harvested bone marrow or peripheral blood apheresis product and from formulation and pre-transplant release testing. Losses of HSC that can be reduced include the loss of long-term repopulating potential in ex vivo culture for genetic manipulation and the loss to peripheral organs during homing that reduce GM HSC engraftment.

Gene therapy has largely failed unless a significantly higher number of GM HSPC are infused than would be for non-gene therapy transplants (Biffi et al., Science, 2013; Aiuti et al., Science, 2013; Kang et al., Blood, 2010; Marktel et al., Nat Med, 2019). Vector integration analysis has shown that long-term repopulation comes from only a very small fraction of the GM HSC transplanted (Kang et al., Blood, 2010; Marktel et al., Nat Med, 2019). When bone marrow cells are infused IV, only 5-30% of HSC home to the bone marrow while the remainder are lost to the periphery, particularly the lung, liver, and spleen (van der Loo et al., Blood, 1995; van Hennik et al., Blood, 1999). Homing losses are further compounded in gene therapy transplants by the lack of helper cells, which are removed during HSPC enrichment. These helper cells aid in the homing and engraftment process.

Herein, using a human NSG xenograft model, direct bone marrow transplantation of GM HSPC enhanced HPC engraftment but did not significantly improve engraftment of LTRC, and the underlying mechanism was shown. The mechanism was utilized by demonstrating transient CXCR4 upregulation in a clinically translatable LTRC population to specifically increase GM LTRC engraftment when transplanted IV and remarkably enhance engraftment of GM LTRC when transplanted IBM. Thus, the compositions and methods disclosed herein would allow for improvement of the homing and/or engraftment of genetically modified (GM) HSCs such as adult HSCs to achieve high levels of engraftment with a limited cell dose, which could broaden the use of gene therapy.

### I. Hematopoietic Cells with Improved Homing and Engraftment Activity

In some aspects, the present disclosure provides hematopoietic cells having improved homing and/or engraftment capabilities post transplantation (e.g., long-term engraftment capability). In some embodiments, such hematopoietic cells may be modified to present one or more exogenous cell homing molecules. The hematopoietic cells disclosed herein may also be genetically modified to carry one or more transgenes of interest (e.g., a gene coding for a therapeutic agent).

In some embodiments, the hematopoietic cells disclosed herein may be a heterogeneous population comprising cells with different features. Such a heterogeneous population, as a whole, displays features of interest, e.g., presence of the exogenous cell homing molecules carrying the transgenes of interest; and/or displaying desired cell surface markers. Alternatively, the hematopoietic cells disclosed herein may be a substantially homogeneous population, for example, at least 75% (e.g., at least 80%, at least 85%, at least 90%, at least 95%, or above) of the cells in the population display the same surface marker(s) and/or possess the same type of modifications as disclosed herein.

### A. Type of Hematopoietic Cells

Hematopoietic cells are cells related to blood cells, including hematopoietic progenitor and stem cells (HPSC), hematopoietic progenitor cells (HPC), and/or hematopoietic stem cells (HSC). The hematopoietic cells disclosed here may be isolated from one or more donor subjects. For example, the hematopoietic cells may be derived from bone marrow, peripheral blood, or umbilical cord blood.

In some embodiments, the hematopoietic cells disclosed herein comprise HPSC cells, which are primarily CD34+cells. In some examples, a population of hematopoietic cells may comprise at least 80% CD34+cells (e.g., at least 85%, at least 90%, at least 95%, or above). HPSCs are primarily found in their niche in the bone marrow and can be released from the niche when induced.

In some embodiments, the hematopoietic cells disclosed herein comprise HPC cells, which are primarily CD34⁺ and CD38⁺cells. HPCs are cells that are capable of multiplying and producing additional blood cells of a particular lineage (e.g., erythroid, myeloid, or megaloblastic). In some examples, a population of hematopoietic cells may comprise at least 80% CD34⁺ and CD38⁺cells (e.g., at least 85%, at least 90%, at least 95%, or above). HPCs can be found in adult bone marrow, peripheral blood, and umbilical cord blood. HPCs can be used to replace or rebuild a patient's hematopoietic system and thus benefit the treatment of many hematopoietic malignant (e.g., leukemia, lymphoma) and non-malignant (e.g., sickle cell disease) diseases.

In some embodiments, the hematopoietic cells disclosed herein comprise HSC cells, which are primarily CD34⁺ and CD38⁻cells. In some instances, the HSC cells are CD34⁺, CD38⁻, and CD90⁺cells. HSCs, which can be found in adult bone marrow, peripheral blood, and umbilical cord blood, are multipotent, self-renewing progenitor cells that develop from mesodermal hemangioblast cells. HSCs can give rise to different types of blood cells, for example, myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells. Like HPCs, HSCs could also help replace or rebuild a patient's hematopoietic system and thus benefit the treatment of many hematopoietic malignant (e.g., leukemia, lymphoma) and non-malignant (e.g., sickle cell disease) diseases. HSCs can also be used for treating autoimmune diseases and hereditary diseases.

In some examples, the hematopoietic cells disclosed herein may comprise a substantially pure population of HSC cells or long-term repopulating cells (LTRCs). For example, at least 80% of the cells are CD34⁺ and CD38⁻cells (e.g., CD34⁺/CD38⁻/CD90⁺), e.g., at least 85%, at least 90%, at least 95%, or above. Alternatively or in addition, such hematopoietic cells may be substantially free of CD34⁺ and CD38⁻cells. In some instances, the hematopoietic cells contain less than 10% CD34⁺/CD38⁻cells (e.g., less than 8%, less than 5%, less than 3% or less than 1%). As reported herein, such a substantially pure HSC/LTRC cell population exhibits improved homing and engraftment activity (e.g., via direct bone marrow delivery) as compared with a cell population having both HSC/LTRC and HPC cells.

In some examples, any of the hematopoietic cell populations (CD34⁺) may be further enriched with CD38⁻, CD90⁺, CD45RA⁻, CD49F⁺, and/or CD133⁺cells.

### B. Cell Homing Molecules

In some embodiments, any of the hematopoietic cell populations disclosed herein comprise modified HPSCs, HSCs, LTRCs, and/or HPCs. Such modified hematopoietic cells may present one or more exogenous cell homing molecules, which may be displayed on cell surface, to improve stem cell homing and/or engraftment activity (e.g., long-term engraftment). As used herein, “an exogenous molecule” refers to a molecule introduced into a cell and not originated from the cell. For example, the one or more exogenous cell homing molecules can be delivered into the hematopoietic cells via protein delivery (see relevant disclosures therein). The hematopoietic cells thus modified present (e.g., displays on surface) the exogenous cell homing molecules but do not contain transgenes producing such cell homing molecules.

Cell homing molecules (also known as homing receptors) are cell adhesion molecules expressed on hematopoietic cell surface that recognize their binding ligands on target tissues. Homing molecules help circulating hematopoietic cells to accumulate at the target tissue, for example, for HSCs to home to the bone marrow after transplantation. Exemplary cell homing molecules include, but are not limited to, CXCR4, or an α4 integrin such as α4β7 and α4β1, VLA-4, CD44, CXCR3, CCR5, E-/P-selectin, MMP2, MMP9, CD26, or LFA-1. In some examples, the cell homing molecule disclosed herein is a CXCR4 polypeptide.

### CXCR4 Polypeptides

As reported herein, an elevated level of CXCR4 expression on HPC cells contributed to the homing and engraftment advantage of HPC cells over HSC cells, which express a lower level of CXCR4. Accordingly, modifying hematopoietic cells such as HSCs or LTRCs with a CXCR4 molecule would be expected to enhance the homing and engraftment activity of the cells thus modified.

C-X-C chemokine receptor type 4 (CXCR-4), also known as fusin or CD184, is a CXC chemokine receptor expressed on many types of cells, including hematopoietic cells and endothelial cells. In humans, CXCR4 is encoded by the CXCR4 gene. Moriuchi et al., J. of Immunology, 159(9):4322-4329 (197). Structural information of human CXCR4 and the CXCR4 gene can be found under Gene ID: 7852.

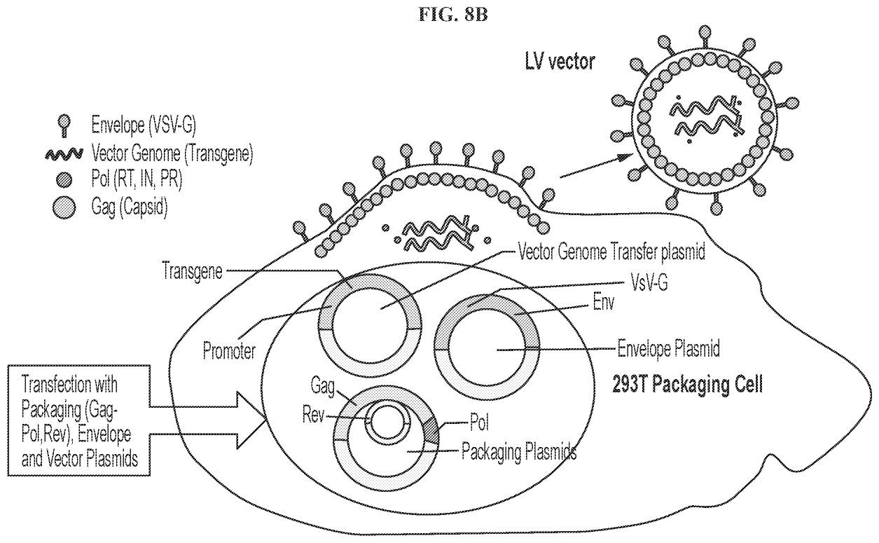

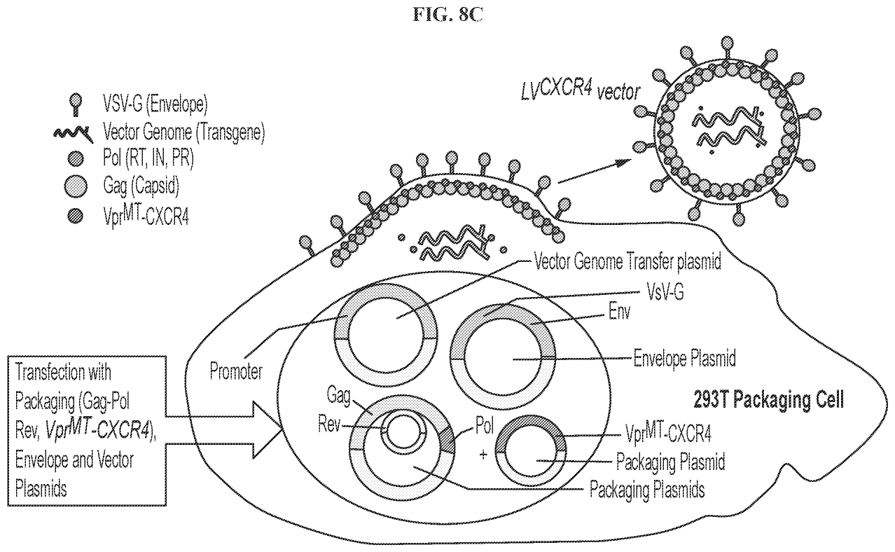

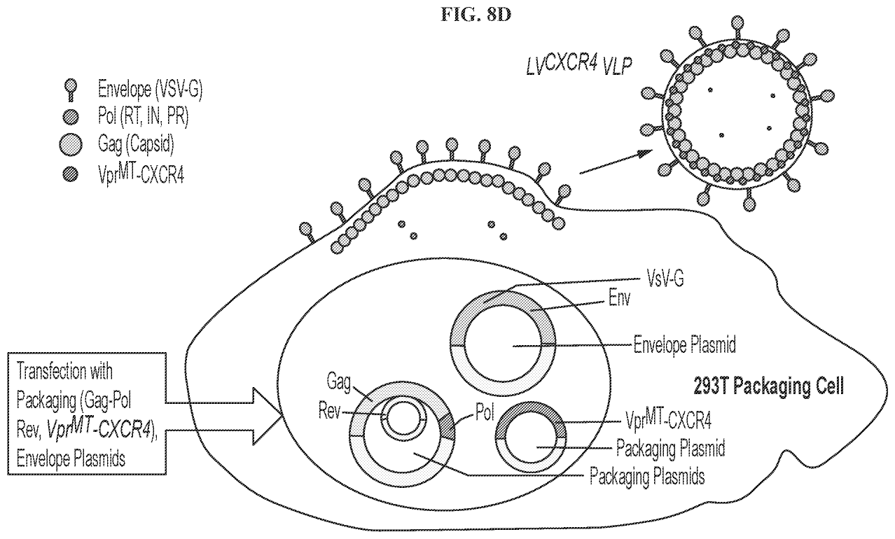

In some embodiments, the CXCR4 protein for use in modifying the hematopoietic cells can be fusion protein comprising a CXCR4 polypeptide fused to a viral protein R (Vpr) or a fragment thereof. In the fusion protein, the CXCR4 portion may be located N-terminal to the Vpr fragment. Alternatively, the CXCR4 portion may be located C-terminal to the Vpr fragment. In some examples, the CXCR4 and the Vpr fragments are linked directly. Alternatively, the two portions may be connected via a peptide linker. A schematic illustration of one exemplary CXCR4-Vpr fusion protein is provided in FIG. 8A.

Vpr is a small HIV-1 accessory protein presented in HIV-1 virions via binding to the capsid. It mediates early T cell toxicity upon cell entry. The structural information of Vpr, including the domains and residues that mediate its toxicity (e.g., cell cycle arrest, apoptosis, and interference of DNA damage response) are well known in the art. See discussions in Examples below. The amino acid sequence of the wild-type Vpr (containing 96 amino acid residues) is provided in FIG. 8A (SEQ ID NO: 1).

The Vpr fragment for use in making the CXCR4 fusion can be a mutated version devoid of the toxicity mediated by the naturally-occurring Vpr. For example, the carboxyl terminal region (e.g., residues 79-96) may be truncated. Alternatively or in addition, residues W54, Q65, and/or R77 in SEQ ID NO:1 may be mutated (e.g., deleted or substituted). Exemplary amino acid substitution at these positions include W54R, Q65R, and/or R77Q.

In some examples, the Vpr portion in the CXCR4-Vpr fusion protein may comprise an amino acid sequence at least 80% (e.g., at least 85%, at least 90%, at least 95%, at least 98%, or higher) identity to residues 1-78 of SEQ ID NO:1. Such a Vpr fragment may contain one or more of the truncation/mutations disclosed herein. In addition, the Vpr fragment may contain one or more conservative amino acid residue substitutions relative to residues 1-78 of SEQ ID NO:1. In one example, the Vpr fragment for use in making the CXCR4-Vpr fusion protein may comprise (e.g., consist of) the amino acid sequence shown in FIG. 8A (SEQ ID NO:2).

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Any of the CXCR4-Vpr fusion protein disclosed herein may further comprises a protease cleavage peptide, which may be located between the CXCR4 and Vpr fragments. Cleavage at the protease cleavage peptide via a suitable protease (which recognizes the cleavage site in the cleavage peptide) would release the CXCR4 polypeptide from the fusion protein, thereby facilitating localization of the CXCR4 polypeptide to cell surface. When lenvirival particles are used for delivering the CXCR4-Vpr fusion protein to host cells, the protease cleavage peptide may comprise a cleavage site recognizable by a lentiviral protease. For example, the cleavage peptide may comprise a polybasic cleavage site (PCS).

Any of the CXCR4-Vpr fusion polypeptides as disclosed herein, as well as nucleic acids encoding such (e.g., vectors such as expression vectors) and host cells comprising the nucleic acids, is also within the scope of the present disclosure.

### C. Genetic Modifications

In some embodiments, any of the hematopoietic cells disclosed herein are genetically modified (GM), for example, carrying one or more transgenes of interest. In some instances, the one or more transgenes encode and can produce one or more therapeutic agents. The therapeutic agent may be a therapeutic protein, for example, an antibody, a growth factor, a cytokine, a coagulation factor, an enzyme, or a hemoglobin. In some examples, the transgenes of interest are provided in US Application No. 2011/0294114A1. In some embodiments, the gene encoding an agent of interest is β-globin or γ-globin, which can be used for treating anemia, e.g., sickle cell anemia or β-thalassemia.

In some instances, the genetic modification may be mediated by a vector such as viral vector. A “vector”, as used herein is any nucleic acid vehicle (DNA or RNA) capable of facilitating the transfer of a nucleic acid molecule into host cells (e.g., hematopoietic cells such as HSCs). In general, vectors include, but are not limited to, plasmids, phagemids, viral vectors, and other vehicles derived from viral or bacterial sources that have been manipulated by the insertion or incorporation of a target nucleotide sequence.

A viral vector contains elements derived from a viral genome (naturally-occurring or modified) and can be used to deliver genetic materials (e.g., a transgene) into suitable host cells. Viral vectors may be based on non-cytopathic eukaryotic viruses in which nonessential genes have been replaced with a target nucleotide sequence. Non-cytopathic viruses include retroviruses (e.g., lentivirus), the life cycle of which involves reverse transcription of genomic viral RNA into DNA with subsequent proviral integration into host cellular DNA. Non-limiting examples of viral vectors include, but are not limited to, retroviral vectors (e.g., lentiviral vectors or gammaretroviral vectors), adenoviral vectors, adeno-associated viral vectors (AAV), and hybrid vectors (containing components from different viral genomes). Additional examples of viral vectors are provided in U.S. Pat. Nos. 5,698,443, 5,650,309, and 5,827,703, the relevant disclosures of each of which are herein incorporated by reference for the purpose and subject matter referenced herein.

In some examples, a retroviral vector can be used to introduce genetic modifications to the hematopoietic cells disclosed herein. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are known in the art.

Other vectors include non-viral plasmid vectors, which have been extensively described in the art and are well known to those of skill in the art. See, e.g., Sambrook et al. Molecular Cloning: A Laboratory Manual. Cold Spring Harbor Laboratory Press; 4th edition (Jun. 15, 2012). Exemplary plasmids include pBR322, pUC18, pUC19, pRC/CMV, SV40, and pBlueScript. Other plasmids are well known to those of ordinary skill in the art.

In some examples, the genetically modified hematopoietic cells may also present one or more cell homing molecules as disclosed herein, for example, any of the CXCR4-Vpr fusion proteins as disclosed herein.

### D. Preparation of Populations of Hematopoietic Cells

To prepare the population of hematopoietic cells disclosed herein, a suitable parent population of hematopoietic cells such as HSPCs, HPCs, or HSC/LTRCs, can be obtained from a suitable source. In some instances, the population of hematopoietic cells as disclosed herein can be derived from a human subject, e.g., from the bone marrow cells, peripheral blood cells, and/or umbilical cord blood cells of the human subject, via a convention method. A specific subpopulation of the stem cells, e.g., CD34+cells, CD34+/CD38+cells, CD34+/CD38−cells, and/or CD34+/CD38−/CD90+cells, may be isolated from the parent population via a conventional methods, e.g., via positive or negative selection.

The isolated hematopoietic cells may be cultured in a suitable medium comprising components for maintaining stemness (e.g., cell self-renewal capability), for example, suitable growth factors, nutritional factors, etc., following routine practice. Any of the modifications (e.g., introduction of the cell homing molecule and/or genetic modification) may be performed during cell culturing. In some instances, the cell homing molecule and the transgene of interest may be delivered to the hematopoietic cells concurrently, e.g., via the virus-like particles disclosed herein.

The hematopoietic cell population thus prepared may be used directly in cell transplantation to a subject in need of the treatment. In some instances, the cell transplantation may be performed to the human patient from whom the hematopoietic cells are derived (autologous therapy). In other instances, the cell transplantation may be performed to a different human patient (allogeneic therapy). Alternatively, the hematopoietic cells may be suspected in a cryopreservation solution and stored (e.g., at −80° C. or in liquid nitrogen) for future use.

### II. Compositions and Methods for Delivering a Proteins of Interest to Host Cells

In some aspects, provided herein are compositions, systems, and methods for delivering a protein of interest such as a cell homing molecule as those disclosed herein at the protein level to a host cell such as a hematopoietic cell as also disclosed herein. In some embodiments, a transgene (e.g., via a viral vector) may be delivered to the host cell (e.g., the hematopoietic cell) concurrently.

### A. Virus-Like Particles

In some embodiments, the protein delivery, optionally together with a transgene, may be achieved by a virus-like particle (VLP). A VLP closely resembles the structure of a virus but is not infectious for lacking essential viral genetic materials necessary for virus proliferation and infection. The VLP disclosed herein comprises a protein shell comprising one or more viral surface proteins, for example, envelope proteins and/or capsid proteins, and one or more cell homing molecules. In some instances, the VLP can further comprise a nucleic acid, which is encapsulated by the protein shell. The nucleic acid may comprise one or more transgenes of interest, which may comprise coding sequence(s) for a therapeutic agent(s), as well regulatory elements controlling and/or regulating expression of the therapeutic agents.

The compositions, systems, and methods disclosed herein can be used for delivering any protein of interest to host cells. The protein of interest may be fused with a viral protein fragment such as the Vpr fragment disclosed herein. In some instances, a protease cleavage site may be located between the protein of interest and the viral protein fragment.

In some embodiments, the protein of interest can be a cell homing molecule as disclosed herein. In some examples, the cell homing molecule is a CXCR4 protein, such as the CXCR4-Vpr fusion polypeptide disclosed herein. The CXCR4 protein can be associated with the surface protein shell formed by viral surface proteins. After the VLP enters a hematopoietic cell, the CXCR4 protein can be delivered to the hematopoietic cell, for example, displayed on cell surface. In some examples, the VLP further comprises a nucleic acid, which can be a viral vector (e.g., a double-stranded DNA molecule) or a nucleic acid (e.g., an RNA molecule or a single-strand DNA) produced from a viral vector. In some instances, the VLP may further comprise a polymerase and other protein components for reverse transcription and/or replication of the nucleic acid contained therein. Such polymerase and other protein components may be associated with the nucleic acid and encapsulated by the protein shell. Alternatively or in addition, the VLP may further comprise a protease that recognizes a protease cleavage peptide located in the fusion protein.

In some instances, the VLP is a lentivirus-like particle, in which the protein shell comprises an envelope protein (e.g., gp120 and/or gp41), a capsid protein (p24 and/or p7/p9)), a matrix protein (e.g., p17), or a combination thereof. The cell homing molecule such as the CXCR4-Vpr fusion polypeptide can be associated with the viral surface proteins in the protein shell. The lentivirus-like particle may further comprise an RNA molecule, which carry a transgene and lentiviral elements for viral particle assembly but no essential viral genes (e.g., gag, pol, etc.) for virus proliferation and infection. Gene products of the Pol gene may be associated with the RNA molecule and encapsulated by the protein shell. Exemplary lentivirus-like particles, with or without nucleic acids, are provided in FIGS. 8C to 8D.

### B. Systems and Methods for Producing VLPs

The present disclosure also provides a system for producing the VLPs disclosed herein for protein delivery of one or more proteins of interest (e.g., one or more cell homing molecules) to host cells (e.g., hematopoietic cells), optionally concurrently with a transgene. Such a system may comprise (i) an expression vector for producing the cell homing molecule (e.g., a CXCR4-Vpr fusion protein disclosed herein), and (ii) a packaging cell line for VLP assembly. In some instances, the packaging cell line is genetically modified to contain all gene materials for producing viral proteins necessarily for VLP assembly (e.g., gag, env, and pol for assembly of a lentivirus-like particle). Alternatively, the system contains (iii) one or more expression vectors for producing such viral proteins. In some examples, the system may further comprise (iv) a viral vector comprising a transgene gene of interest and viral elements for packaging the viral genome-like nucleic acid produced from the viral vector to the VLP.

### Retroviral Vector

In some examples, the system disclosed herein comprise a retroviral vector for genetic modification of a hematopoietic cell, e.g., delivering a transgene of interest. A retroviral vector is a DNA molecule containing proviral sequences (e.g., LTR sequences, Psi (ψ) sequence, and/or promoter/enhancer sequence) that can accommodate a gene of interest, to allow incorporation of both into target cells. The proviral sequences are derived from a retroviral genome and are modified such that they can be used as a plasmid vehicle for carrying and transferring genetic materials. The proviral sequences are also modified to remove essential viral genes and safety concerns. Typically, a retroviral vector is incapable of self-proliferation and/or packaging to produce viral particles without presence of helper virus that provides essential viral proteins/genes.

Retroviruses include 7 families: alpharetrovirus (Avian leucosis virus), betaretrovirus (Mouse mammary tumor virus), gammaretrovirus (Murine leukemia virus), deltaretrovirus (Bovine leukemia virus), epsilonretrovirus (Walleye dermal sarcoma virus), lentivirus (Human immunodeficiency virus 1), and spumavirus (Human spumavirus). Six additional examples of retroviruses are provided in U.S. Pat. No. 7,901,671. Viral elements, such as those described herein, from a suitable retrovirus can be used to construct the retroviral vectors described herein. The retroviral vectors described herein may be a lentiviral vector or a gammaretroviral vector. Non-limiting examples of retroviral vectors include human immunodeficiency viral (HIV) vector, avian leucosis viral (ALV) vector, murine leukemia viral (MLV) vector, murine mammary tumor viral (MMTV) vector, murine stem cell virus, and human T-cell leukemia viral (HTLV) vector. These retroviral vectors comprise proviral sequences from the corresponding retrovirus.

The retroviral vector described herein comprises a 5′ long terminal repeat (LTR), a 3′LTR, and any of the insulator fragments described herein, which may be inserted into one or both of the LTR regions. In addition, the retroviral vector may comprise additional viral or non-viral elements to facilitate the intended viral vector functionality as described herein. The LTR regions are typically located on opposite ends of a retroviral vector, which can be a linear DNA molecule. In some embodiments, the LTRs of the retroviral vector comprise a U3 region, a R region, and a U5 region. In some instances, the U3 region in the 5′ LTR, the 3′ LTR or both may comprise enhancer/promoter elements, which may drive the expression of genes within the retroviral vector. These enhancer/promoter elements may function as either an enhancer, a promoter, or both. Such retroviral vectors are often referred to as LTR-driven vectors (Maetzig et al., Viruses 3(6):677-713, 2011). In other instances, the 5′ LTR, the 3′ LTR, or both may have one or more of the U3 region, the R region, and the U5 region deleted (e.g., self-inactivated vectors such as those described below).

The retroviral vectors described herein may further comprise additional functional elements as known in the art to address safety concerns and/or to improve vector functions, such as packaging efficiency and/or viral titer. Additional information may be found in US20150316511 and WO2015/117027, the relevant disclosures of each of which are herein incorporated by reference for the purpose and subject matter referenced herein.

The retroviral vectors described herein can be prepared by conventional recombinant technology. In some examples, an insulator fragment as those described herein may be inserted into a suitable location of a retroviral vector to reduce genotoxicity of the resultant retroviral vector. For example, the insulator fragment may be inserted inside the 5′ LTR, inside the 3′ LTR, or inside both the 5′ LTR and the 3′ LTR via conventional technology. When desired, additional insulator fragments can be inserted at suitable sites inside the retroviral vector, for example, adjacent to a transgene carried by the retroviral vector. As used herein, the term “inserting” refers to the process of adding a sequence of nucleotides to the retroviral vector by using, for example, restriction digestion and ligation or recombination. Techniques for inserting sequences into retroviral vectors would be apparent to those skilled in the art.

In conventional gene therapy, self-inactivating (SIN) GV and LV vectors with a 3′LTR have been used increasingly to circumvent the risk of insertional oncogenesis by viral enhancers. These SIN GV and LV vectors have U3 enhancer/promoter deletion and internal, weaker cellular/endogenous gene promoters driving transgene expression. This deletes ubiquitously active enhancers in the U3 region of the long terminal repeats (LTR). These SIN ‘LTR-less’ or ‘enhancer-less’ vectors show reduced genotoxicity as compared to LTR-intact GV vectors in experimental systems both in vitro and in vivo (Modlich et al., Blood 108:2545-53, 2006, Zychlinski et al., Mol. Ther. 16:718-25, 2008, Montini et al., J Clin Invest 119:964-75, 2006). However, expression of the transgene is often not robust, and successful and complete correction of the disease phenotype is largely dependent on introduction of high numbers of transduction/vector copy number (VCN) per cell, except in diseases where modest levels of transgene expression are sufficient for correction.

### VLP Production

In some instances, any of the nucleic acid constructs described herein (e.g., expression vectors for producing the cell homing molecule, expression vectors for producing viral proteins, and/or the viral vectors such as retroviral vectors) can be transfected into suitable packaging cells for producing viral particles. Techniques for transduction of nucleic acid construct into host cells such as into mammalian cells are well established in the art. Some examples are provided in U.S. Pat. No. 5,399,346. Methods of nucleic acid transfection are well established in the arts and range from chemical, to biological, and to physical methods. Chemical methods include, but are not limited to, calcium phosphate transfection, cationic polymer transfection, lipofection, FUGENE®, and DEAE-Dextran-mediated transfection. Other methods of transfection include, but are not limited to, electroporation, sonoporation, cell squeezing, impalefection, optical transfection, protoplast fusion, magnetofection™, and particle bombardment.

When the nucleic acid constructs comprise a viral vector such as a retroviral vector, the host cells can be packaging cells that express viral structural and/or accessory proteins (e.g., retroviral structural and/or accessory proteins), for example, gag, pol, env, tat, rev, vif, vpr, vpu, vpx, and/or nef. Alternatively, such viral structural and/or accessory proteins may be encoded by expression vectors co-introduced into the packaging cells. Viral envelope proteins (env) determine the range of host cells to which the viral particles can infected and transform by recombinant retroviruses generated from the packaging cell lines. In the case of lentiviruses, such as HIV-1, HIV-2, SIV, FIV and EIV, the env proteins include gp41 and gp120. In some instances, a gene coding for the viral env proteins may be on a separate vector as those encoding for viral gag and pol. In other instances, genes coding for env, pol, and gag may be located on the same vector. Such vectors can be transfected into suitable packaging cells for stable expression of the viral proteins.

Packaging cells do not contain a packaging signal in its genetic materials and are capable of expressing (e.g., stably) viral structural proteins, replication enzymes (e.g., gag, pol, and env), as well as others that are necessary for the packaging of viral particles. Any suitable cell lines, for example, mammalian cell lines, can be employed to prepare packaging cells. Examples include CHO cells, BHK cells, MDCK cells, COS cells, VERO cells, 3T3 cells, NIH3T3 cells, HepG2 cells, HeLa cells, 293 cells, 293T cells, or A549 cells.

Methods of preparing viral stock solutions from packaging cells are known in the art and are illustrated by, e.g., Y. Soneoka et al., Nucl. Acids Res. 23:628-633, 1995 and N. R. Landau et al., J. Virol. 66:5110-5113, 1992. Infectious virus particles may be collected from the packaging cells using conventional techniques. For example, the infectious particles can be collected by cell lysis, or collection of the supernatant of the cell culture, as is known in the art. If needed, the collected virus particles may be purified using conventional technology.

### C. Modification of Hematopoietic Cells

The VLPs thus produced, which comprise the cell homing molecule in the protein shell, and optionally a nucleic acid such as an RNA molecule transcribed from any of the retroviral vectors described herein, can be used to infect any of the hematopoietic cells disclosed herein, thereby modifying the hematopoietic cells (e.g., delivering the cell homing molecule and optionally the transgene carried by the nucleic acid). In some examples, the cell homing molecule delivered to the hematopoietic cell is a CXCR4 protein such as a CXCR4-Vpr fusion polypeptide as disclosed herein.

The VLPs can be brought in contact with the hematopoietic cells in cell culture to allow for entry of the VLPs into the hematopoietic cells. The resultant modified hematopoietic cells can then be used for cell transplantation and gene therapy. In some instances, the hematopoietic cells may be characterized for confirming presence of the cell homing molecule and optionally the transgene or the therapeutic agent produced thereby.

The VLP particles disclosed herein, carrying the cell homing molecule, can be used to deliver the cell homing molecules to other types of cells, for example, immune cells such as T cells and/or NK cells. A transgene encoding a chimeric antigen receptor (CAR) may be co-delivered with the cell homing molecule to produce CAR-expressing immune cells having the exogenous cell homing molecule, which could enhance homing of such immune cells after transplantation.

### III. HSC Transplantation and Gene Therapy

Any of the hematopoietic cells (e.g., HSPC, HPC, or HSC/LTRC) having enhanced homing and/or engraftment capacities as disclosed herein may be used in stem cell therapy and gene therapy for treatment of a target disease.

Exemplary target diseases include, but are not limited to, neurodegenerative diseases and conditions, diabetes, heart disease, blood disorders, immune disorders, and genetic disorders. Examples of suitable conditions to be treated by stem cell therapy and gene therapy include, but are not limited to, acute myeloid leukemia (AML), chronic myeloid leukemia (CML), acute lymphoblastic leukemia (ALL), Hodgkin lymphoma, Non-Hodgkin lymphoma, neuroblastoma, Ewing sarcoma, Myelodysplastic syndromes, Gliomas, and other solid tumors. Stem cell therapy can also be applied to non-malignant conditions such as thalassemia, aplastic anemia, Fanconi anemia, immune deficiency syndromes, or inborn errors of metabolism. In some embodiments, the HSCs prepared by the ex vivo culturing methods described herein can be used for transplantation in treatment of hematopoietic disorders, including, but not limited to, acute myeloid leukemia (AML), chronic myeloid leukemia (CML), acute lymphoblastic leukemia (ALL), chronic lymphocytic leukemia (CLL), juvenile myelomonocytic leukemia, Hodgkin lymphoma, and Non-Hodgkin lymphoma.

Hematopoietic stem cell transplantation (HSCT) is the transplantation of hematopoietic stem cells, for example, HSPCs, HPCs, and HSC/LTRCs as disclosed herein, which may be modified as also disclosed herein. In some instances, the hematopoietic cells can be autologous (the patient's own stem cells are cultured by the ex vivo culturing methods described herein and used for treating a disease). In other examples, the hematopoietic cells can be allogeneic (the stem cells come from a donor and is then cultured by the ex vivo culturing methods described herein). Such hematopoietic cells can be used for treating certain cancers of the blood or bone marrow, such as multiple myeloma or leukemia. In these cases, the recipient's immune system is usually destroyed with radiation or chemotherapy before the transplantation.

In any of the stem cell/gene therapy described herein, suitable stem cells as disclosed herein can be collected from the ex vivo culturing method described herein, which may subject to the modification as also disclosed herein, and mixed with a pharmaceutically acceptable carrier to form a pharmaceutical composition, which is also within the scope of the present disclosure.

To perform the treatment methods described herein, an effective amount of the stem cells can be administered into a subject in need of the treatment. In some embodiments, the hematopoietic cells may be administered to the subject via intravenous infusion. Alternatively, the hematopoietic cells may be administered to the subject via direct bone marrow administration (intra-bone marrow injection or IBM). In some examples, a hematopoietic stem cells (CD34+ and CD38−) substantially free of CD34+ and CD38+cells can be delivered to a human patient by intra-bone marrow injection.

In one example, a gene therapy method involving genetically modified hematopoietic cells may be performed as follows. A population of hematopoietic cells can be collected from a human subject (e.g., from bone marrow and/or peripheral blood, etc.) via a conventional method. In some instances, a subpopulation of stem cells (e.g., CD34+, CD34+/CD38+, CD34+/CD38−, CD34+/CD38−/CD90+, or a combination thereof) may be isolated. The population of hematopoietic cells or the subpopulation thereof may subject to modifications by instruction of a cell homing molecule (e.g., a CXCR4 protein such as a CXCR4-Vpr fusion protein) and a transgene encoding a therapeutic agent. The hematopoietic cells or the subpopulation thus modified can then be administered to a human patient in need of the treatment via, e.g., intravenous infusion or intra-bone marrow injection. In some instances, the cells are derived from the same human patient who receives the treatment.

In some embodiments, the hematopoietic cells as disclosed herein can be co-used with a therapeutic agent for a target disease, such as those described herein. The efficacy of the stem cell/gene therapy described herein may be assessed by any method known in the art and would be evident to a skilled medical professional. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1: DIFFERENTIAL CXCR4 EXPRESSION ON HEMATOPOIETIC PROGENITOR CELLS VERSUS STEM CELLS DIRECTS HOMING AND LONG-TERM ENGRAFTMENT

Gene therapy involves a substantial loss of hematopoietic stem and progenitor cells (HSPC) during processing and homing. Intra-bone marrow (IBM) transplantation can reduce homing losses but prior studies have not yielded promising results. This study explores the mechanisms involved in homing and engraftment of IBM-and IV-transplanted gene-modified (GM) human HSPC.

### Methods

### Human CD34+HSPC Isolation, Culture, and Transduction

UCB and G-CSF MPB were obtained and CD34+HSPC were isolated by magnetic selection with Indirect CD34 Microbead kit, human (Miltenyi, 130-046-701). CD34+purity was >95% as confirmed by flow cytometry. CD34+CD38−cells were isolated from G-CSF MPB by magnetic selection with CD34+CD38−Isolation Kit, human (Miltenyi, 130-114-822) and cryopreserved. Thawed CD34+ or CD34+CD38−cells were cultured in X-VIVO 10 (Lonza BE02-055Q) or SCGM (CellGenix 20806-0500) supplemented with 2% human serum albumin, 100 ng/mL TPO, 300 ng/mL SCF, and 300 ng/mL FLT3-L (all cytokines purchased from Peprotech) at a cell density of 2-5×10⁶ CD34+cells/mL. Media was supplemented with Birb 796 (600 nM) (Selleckchem) throughout culture and Prostaglandin E2 (10 μM) (Cayman) at plating, transduction, and 1 hour before harvest. Cells were transduced with a lentiviral vector in culture for 36-42 hours where indicated. CD34+HSPC were transduced at a final concentration of 5×10⁷-1×10⁸ IU/mL. Following culture, CD34+HSPC were harvested and washed with PBS, resuspended in PBS, and used in in vivo or in vitro experiments, as described below.

### Mouse Xenograft Model

6- to 14-week-old NOD.Cg-Prkdc^{scid} Il2rg^{tm1Wjl}/SzJ (NSG) mice were used in UCB- and MPB-derived HSPC transplant experiments. Mice were irradiated with 280 cGy prior to transplant. For homing experiments, each mouse received 3×10⁶ CD34+cells and 16-22 hours post-injection, the mice were sacrificed and bone marrow from the femurs was analyzed for human cell content. For long-term engraftment experiments, mice received limiting dilution CD34+or CD34+CD38−HSPC doses. IV injections were done by injecting HSPC in 200-250 μL via tail vein and IBM injections were done by injecting HSPC in 10 μL into the femur.

### Lentiviral Vectors

All LV vectors were packaged using the Gag-Pol, Rev and VSV-G envelope plasmids. All vectors were under the control of the MNDU3 promoter. The GFP LV construct was pRRL.SIN.cPPT.MNDU3.eGFP.WPRE. The BFP LV construct was designed by replacing the GFP cDNA in pRRL.SIN.cPPT.MNDU3.eGFP.WPRE.with mTag2BFP. The BFP-CXCR4 LV was pRRL.SIN.cPPT.MNDU3.mTag2BFP-CXCR4.WPRE. All lentiviral vectors were packaged in HEK 293T (ATCC) cells and vector titers determined in the murine erythroleukemia (MEL) (ATCC) cells as previously described (Perumbeti et al., Blood, 2009; Urbinati et al., Mol Ther, 2009; Arumugam et al., Mol Ther, 2009).

### Protein Delivery of CXCR4 in the Lentiviral Vector

A Vpr-CXCR4 fusion was created by fusing CXCR4 cDNA to a synthesized truncated and mutated Vpr sequence. A 78 amino acid truncated Vpr sequence was designed, lacking the 18 amino acids from the carboxy terminus to remove a major nuclear localization signal and the portion that is critical for Vpr-mediated cell cycle arrest (Barnitz et al., PLoS One, 2011; Marzio et al., J Virol, 1995; Zhou et al., J Virol, 2000). Three point mutations were introduced—Q65R mutation that abrogates its binding to DCAF and is the primary event that triggers a change in the cellular proteome of viral particle delivered Vpr including depletion of cell cycle regulatory proteins and proteins involved in DNA damage response (Greenwood et al., Cell Rep, 2019) and R77Q, and W54R point mutations to further prevent its cytotoxic activities and reduce its stability; these are mutations seen in long-term non-progressors with HIV infection (Guenzel et al., Front Microbiol, 2014; Wallet et al., Biochem Pharmacol, 2020; Soares et al., Rev Med Virol, 2016). This mutated and truncated Vpr (Vpr^{MT}) that retains its ability to bind Gag (the lentiviral capsid protein) but lacks VPR-associated cytotoxicity, was then fused to CXCR4 using the HIV-1 protease cleavage site (Link et al., Nucleic Acids Res, 2006) so that CXCR can be cleaved from Vpr^{MT} by the protease present in the lentiviral particle. This fusion plasmid was used along with the other packaging plasmids to package a GFP-encoding lentiviral vector, using the GFP LV construct listed in Lentiviral vectors.

### Bioluminescent Imaging

Mice received an intra-peritoneal injection of 150 mg/kg body weight D-luciferin (Xenogen XR-1001) 15 minutes before being anesthetized with 3% isoflurane. Anesthetized animals were imaged with the Perkin Elmer In Vivo Imaging System (IVIS) and then allowed to recover fully from anesthesia.

### Secondary Transplants

Bone marrow was harvested from primary transplanted NSG mice at 24 weeks post-transplant. Bone marrow underwent magnetic mouse CD45 antibody depletion using Biotin Rat anti-mouse CD45 (BD Biosciences 553078) and Streptavidin Particles Plus (BD Biosciences 557812) and was transplanted one-to-one IV into irradiated (280 cGy) NSG mice. Secondary mice were analyzed at 12 weeks post-transplant.

### CXCR4 Adhesion Assay

CXCR4 ligand affinity was measured by adhesion assay (Gur-Cohen et al., Nat Med, 2015). Tissue-culture treated flasks were coated with 2.5 μg/mL CXCL12 (Peprotech 300-28A) overnight at 4° C., washed with PBS and then 2×10⁶ CD34+/mL were plated on the CXCL12-coated plate and incubated at 37° C. for 2 hours. Following the 2-hour incubation, non-adherent HSPC were removed. Adherent cells were harvested with rigorous flushing with PBS.

### AMD3100

CD34+HSPC were resuspended in media containing 100 ug/mL AMD3100 (Sigma A5602) for 37° C. for 15 minutes. The cells were washed with PBS and transplanted into NSG mice for homing experiments.

### BIO5192

CD34+HSPC were resuspended in PBS containing 1 μM BIO5192 (Tocris, 5051) and incubated on ice for 20 minutes. The cells were washed with PBS and transplanted into NSG mice for homing experiments.

### Flow Cytometry

For HSC analysis, CD34+cells were stained with the following antibodies: PE-Cy7 Mouse Anti-Human CD34 (BD Pharmingen, 560710) or CD34 APC (BD Pharmingen, 555824), Anti-Human CD38 APC eFluor 780 (eBioscience 47-0389-42), Alexa Fluor 700 anti-human CD90 (BioLegend, 328120), and Anti-Human CD45RA APC (eBioscience 17-0458-42). For VLA-4 staining, PE Mouse anti-human CD49d (BD BioSciences 555503) was used. For CXCR4 staining, PE-Cy7 anti-human CD184 (CXCR4) (BioLegend 306514) was used. For cell cycle staining, cells were stained with cell surface antibodies, fixed using BD Fix and Perm (BD Pharmingen 554714), and then stained with Hoescht-33412 (BD Pharmingen). For Annexin V and 7AAD staining, PE Annexin V Apoptosis Detection Kit with 7-AAD was used (Biolegend 640934). γH2AX was stained with PE γH2AX (Biolegend 613411).

At 12 weeks following transplant, bone marrow was harvested via aspirate of the femur for in vivo engraftment analysis. For animals that received IBM injections into one femur, the non-injected alternate femur was aspirated. For 24 week and homing analysis, mice were sacrificed, and bone marrow was harvested from both the rear and forelimbs. Cells were stained fresh with the following antibodies: PerCP anti-human CD45 (BioLegend 304026), Anti-human CD33 PE-Cy7 (eBioscience 25-0338-42), APC-Cy7 Mouse Anti-Human CD19 (BD 557791), Anti-Human CD3 PE (BD BioSciences 555340), and APC Mouse Anti-Human CD34 (BD BioScience 555824). Red bloods cells were lysed after staining using red blood cell lysis buffer.

Stromal cells were analyzed by staining cells with: Biotin anti-mouse CD51 (Biolegend 104104), FITC anti-mouse CD45 (Biolegend 368508) and Alexa Fluor 700 anti-mouse lineage cocktail (Biolegend 133313).

### Results

IBM HSPC transplantation was found to improve engraftment of hematopoietic progenitor cells (HPC) but not long-term repopulating cells (LTRC). Mechanistically, HPC expressed higher functional levels of CXCR4 than LTRC, conferring them a homing advantage when transplanted IBM. Removing CXCR4high HPC and transplanting an LTRC-enriched population IBM resulted in significantly higher long-term engraftment than IV transplantation. CXCR4 was transiently upregulated on GM LTRC using a non-cytotoxic portion of Vpr fused to CXCR4 in the lentiviral particle, which resulted in higher homing and long-term engraftment of GM LTRC transplanted both IV and IBM compared to standard IV transplant.

Overall, a mechanism for why IBM transplants do not significantly improve long-term engraftment over IV transplants was shown. IBM transplantation becomes relevant when an LTRC-enriched population was delivered. Alternatively, transiently increased CXCR4 expression using a protein delivery method can improve homing/engraftment specifically of GM LTRC transplanted IV to levels comparable with IBM transplants.

### (A) Direct Bone Marrow Transplant Enhanced Hematopoietic Progenitor Cell (HPC) Engraftment, But not Engraftment of the Long-Term Repopulating HSC

Intra-femoral injection was first validated as an accurate means of IBM delivery. UCB CD34+HSPC were transduced with a luciferase-encoding lentiviral vector and transplanted either IV or IBM into irradiated NSG (NOD.Cg-Prkdc^{scid} Il2rg^{tm1Wjl}/SzJ) mice. Bioluminescent imaging was performed 1, 2, and 12 weeks post-transplant, and transduced cell signal was first detected at 2 weeks when sufficient cell proliferation had occurred. IBM injection of HSPC into the femur primarily localized and retained the GM HSPC in the injected femur, while GM HSPC transplanted IV were found in both femurs and in the sternal and rib areas. However, by 12 weeks post-transplant, engraftment of GM cells was widespread with both IV and IBM transplant.

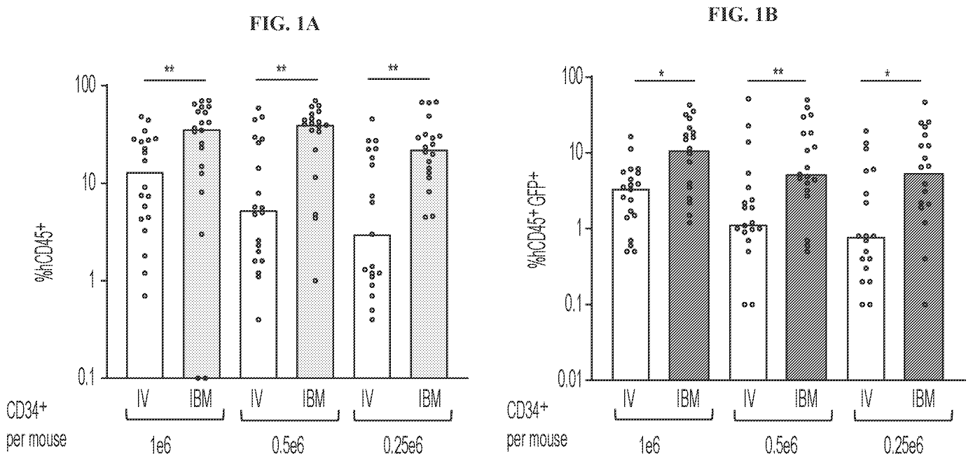

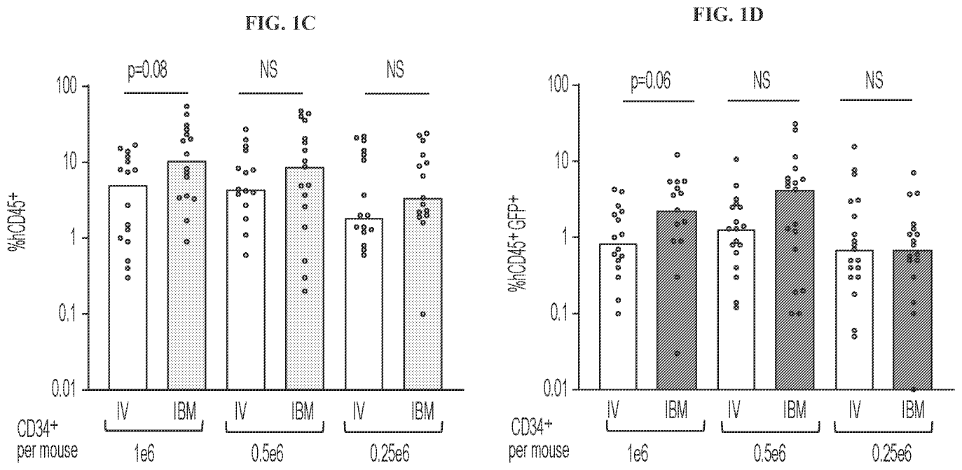

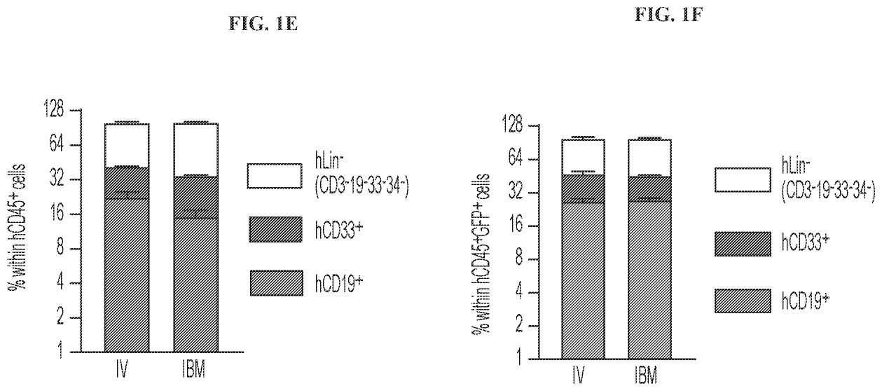

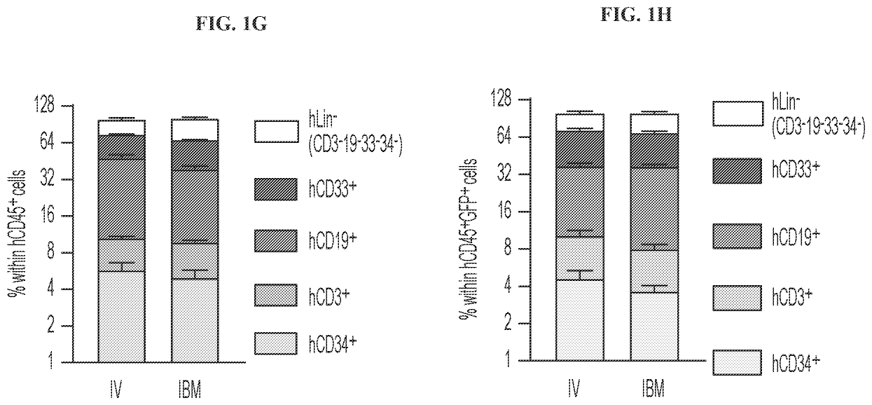

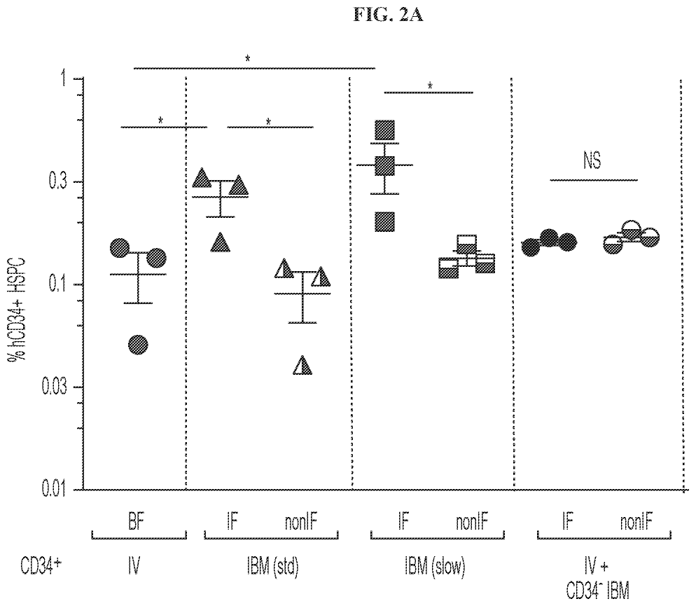

The short-and long-term engraftment of GM adult human HSPC when transplanted via IBM injection compared to the traditional IV transplant was determined. Mobilized peripheral blood (MPB) CD34+HSPC transduced with a GFP-encoding lentivirus vector were transplanted to NSG mice, following the experimental scheme depicted in FIG. 6. At 12 weeks post-transplant, femoral bone marrow aspirates (from the non-injected femur in the IBM group of mice) were performed and analyzed human cell engraftment (human CD45+cells) in the bone marrow and transduced human cell engraftment (human CD45+GFP+). IBM transplantation of HSPC led to significantly higher engraftment of both total and GM human cells in the bone marrow at 12 weeks, particularly when HSPC dose was limited (FIGS. 1A-1B). However, the long-term (24 weeks post-transplant) bone marrow analysis revealed that the engraftment advantage of direct bone marrow transplantation seen at the 12 weeks was lost. While the long-term repopulation trended higher with IBM delivery, the improvement over IV delivery was not significant (FIGS. 1C-1D).

Bone marrow was also analyzed for human multi-lineage repopulation at 12 and 24 weeks. At 12 weeks, the human graft was bi-lineage, primarily composed of B-cells and myeloid cells in both total (FIG. 1E) and transduced (FIG. 1F) human cells, indicating the progeny of a short-term repopulating cell, likely a hematopoietic progenitor cell (HPC). At 24 weeks, a multi-lineage human graft composed of T cells, B cells, myeloid cells and CD34+HSPC was represented in both the total (FIG. 1G) and transduced (FIG. 1H) human graft, indicating that, at this time point, the engraftment represented that of a multi-lineage long-term repopulating cell, likely an HSC.

A secondary transplant of the human graft from primary mice at 24 weeks (after depleting murine CD45+cells) into secondary NSG mice was then performed to definitively compare engraftment of HSC delivered IV versus IBM. There was no observed difference in secondary engraftment of IBM or IV transplanted HSC in secondary mice and data was similar to the long-term engraftment results at 6 months in primary mice (FIG. 11). Hence, the 6 month human primary graft was assumed to be derived from a long term repopulating HSC that gives rise to a multi-lineage graft, capable of secondary engraftment.

Of note, at both 12-week and 24-week time points, the GM HSPC engraftment (FIGS. 1B and 1D) followed patterns similar to that of the overall human HSPC engraftment (FIGS. 1C and 1D). Taken together, the data suggest that IBM transplantation of HSPC provided an engraftment advantage to HPC, but not to the long-term repopulating HSC.

### (B) IBM Transplant Improved Homing of HSPC via an HSPC-Intrinsic Mechanism

Next, this study investigated if there was a specific retention or homing advantage to HPC delivered locally into the bone marrow. It was shown that intra-femoral injection of HSPC in rodents resulted in the majority of cells leaving the bone marrow into circulation within the first 15 seconds but that the injected HSPC home back much more efficiently into the non-injected bone marrow than cells delivered by IV injection (Massollo et al., Exp Hematol. 2010). In a porcine model that better simulates human physiology and scale, Pantin et al. showed that HSPC are retained in the injected bone marrow by lowering the injection volume and injection rate (Pantin et al., Am J Transplant, 2015). Therefore, the retention and homing of HSPC in HSPC in the human engraftment model was determined via four modes of transplant of MPB CD34+HSPC: (1) IV injection of CD34+HSPC by tail vein, (2) IBM delivery of CD34+HSPC, (3) slow IBM delivery of CD34+HSPC over 1 minute using a Hamilton syringe, and (4) a ‘sham’ IBM delivery of irradiated CD34-cells followed by a tail vein injection of CD34+HSPC. The latter was done to determine whether the IBM injection induced mechanical/shear stress that altered the bone marrow microenvironment, allowing superior retention and homing of IV injected cells in the injected femur.

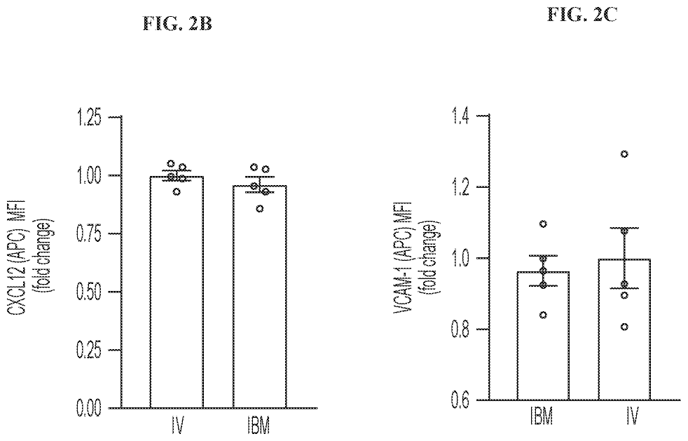

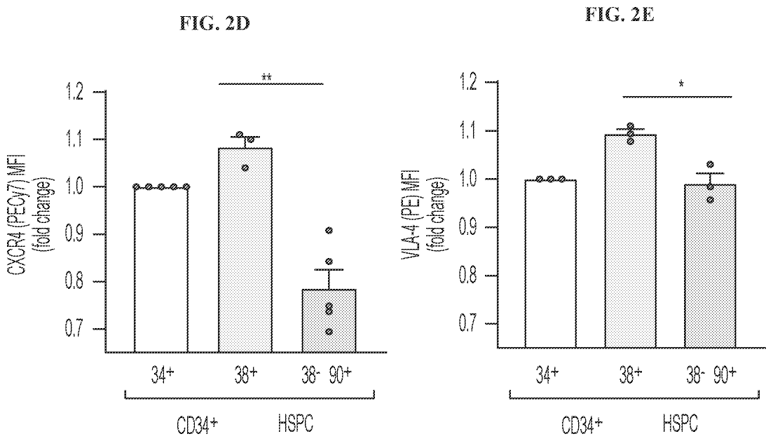

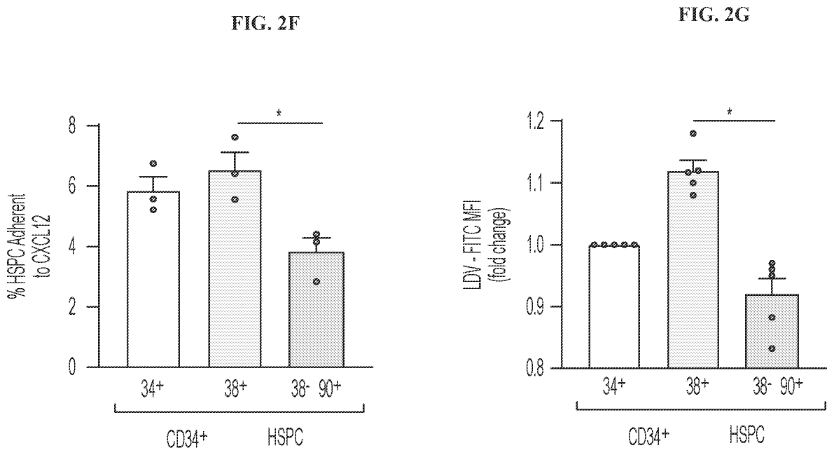

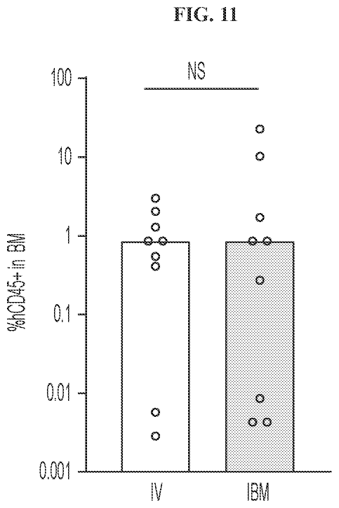

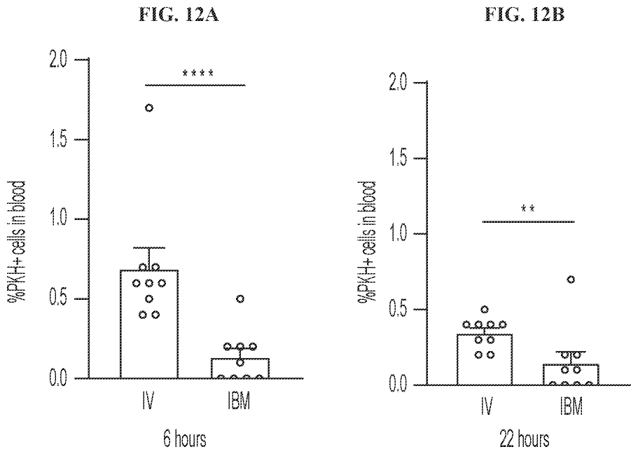

Twenty hours post-infusion, mice were sacrificed. For IBM injections, retention of HSPC was determined in the femurs separately; for IV injections, homing was determined in both femurs combined (FIG. 2A). To determine the accuracy of the injections and explain the homing of IBM injected HSPC to the contralateral femur, CD34+HSPC were also labeled with PKH-26 (so that the populations could be clearly identified in blood without any background seen with antibody staining and analyzed their presence in blood after IV and IBM injections. While a small number of HSPC were in circulation after IBM injections at 6 and 22 hours after transplant, a much higher number of CD34+HSPC were in circulation at 6 hours and 22 hours post IV transplant. See FIGS. 12A and 12B.

The injected femur (IF) of IBM mice had significantly higher human CD34+HSPC content than the non-injected femur (nonIF). The nonIF had similar homing of HSPC into bone marrow as that seen in the IV injected mice. Slow IBM delivery of HSPC did not alter the HSPC patterns as compared to the standard IBM injections, indicating that retention and homing of CD34+HSPC in the IF was not impacted by delivery pressure if a small volume is injected in the mice. Additionally, the sham injection of CD34−cells did not alter homing behavior of IV-injected CD34+HSPC in the IF, which had similar homing as that seen in the non-sham IF, suggesting that a ‘HSPC-intrinsic’ rather than microenvironment mechanism likely mediated this effect (FIG. 2A). Consistent with this observation, the mechanical pressure of IBM delivery did not alter the expression of stromal cell ligands for the two most well characterized homing receptors on HSPC. Bone marrow stromal cells (mouse lineage negative, human CD45−PKH−, mouse CD51+cells; human HSPC were PKH labeled; gating for murine stromal cells from the IF and nonIF were studied to determine if expression of CXCL12, the ligand for CXCR4, and VCAM-1, the ligand for VLA-4, changed in response to pressure increases associated with injection through the bone marrow. Expression of CXCL12 (FIG. 2B) and VCAM-1 examined in the murine and human non-hematopoietic mouse stromal cell population (FIG. 2C) was not significantly different in the bone marrow stroma in the IF compared to the nonIF, further suggesting that microenvironment changes due to injection were not a significant factor in observed differential engraftment behavior, and that the difference was likely cell-intrinsic.

### (C) Differential Homing Receptor Expression on HPC and HSC

It was postulated that cell-intrinsic differences of homing receptor expression between HPC and HSC may explain the differential short-and long-term engraftment. The expression of the two major HSPC homing and retention receptors, CXCR4 and VLA-4, were determined on CD34+CD38+cells, which are largely HPC, and CD34+38−90+cells, a population highly enriched in HSC. CD34+38+HPC had significantly higher surface expression of VLA-4 and CXCR4 than did CD34+38−90+HSC (FIGS. 2D-2E). Accordingly, CD34+38+HPC functionally adhered more avidly to a CXCL12-coated plate than did the CD34+38−90+HSC-enriched cells (FIG. 2F). Furthermore, CD34+CD38+HPC had increased VLA-4-ligand binding affinity than did CD34+38−90+HSC (FIG. 2G). Overall, these results suggested that increased VLA-4 and CXCR4 expression on HPC may be involved in conferring a retention and homing advantage to HPC over HSC following IBM administration, and therefore, the engraftment advantage was short-lived.

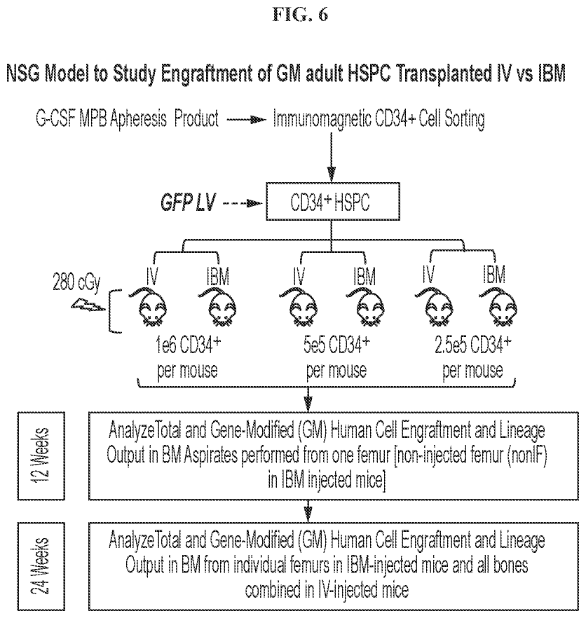

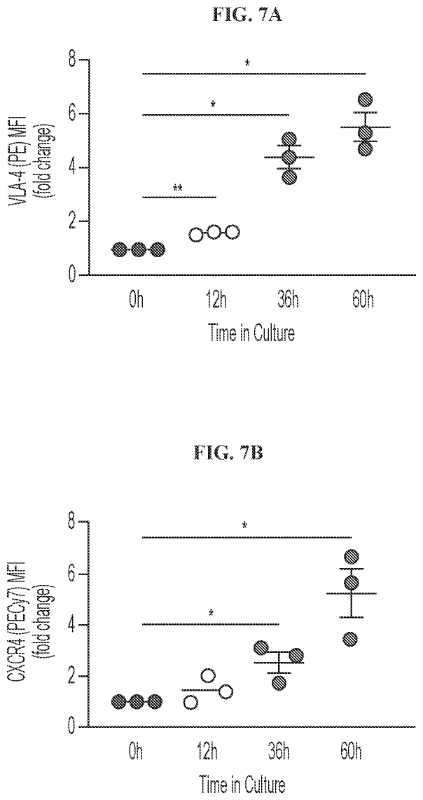

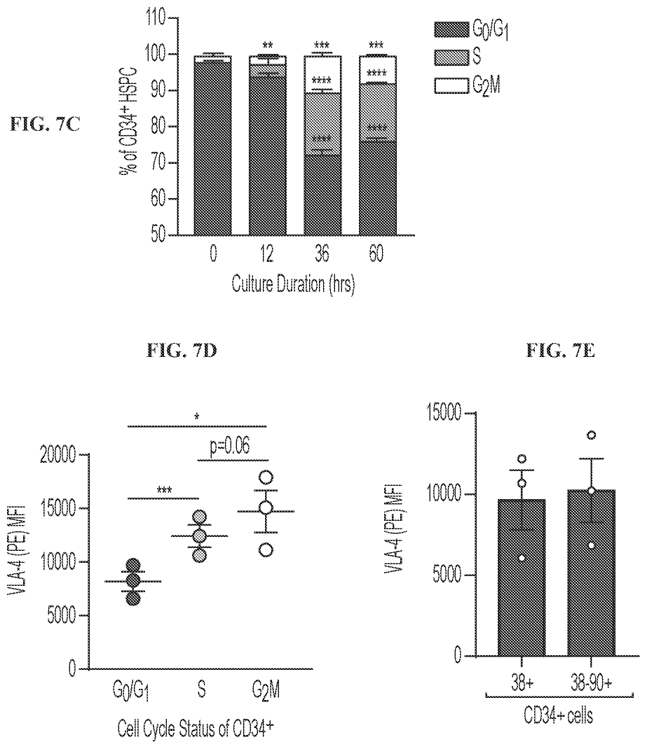

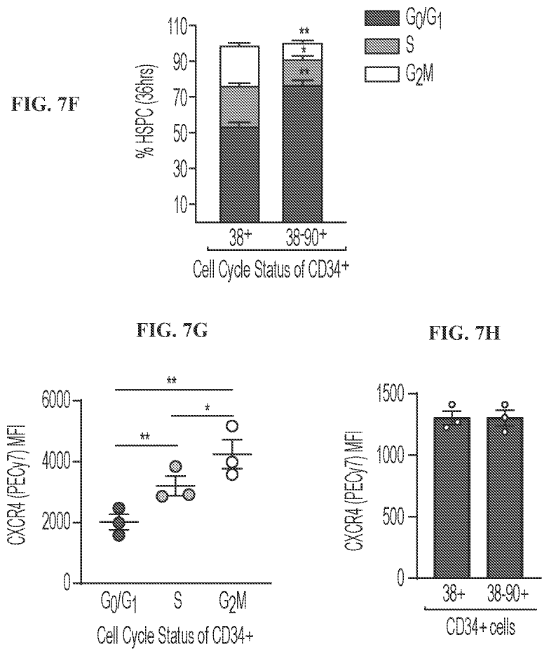

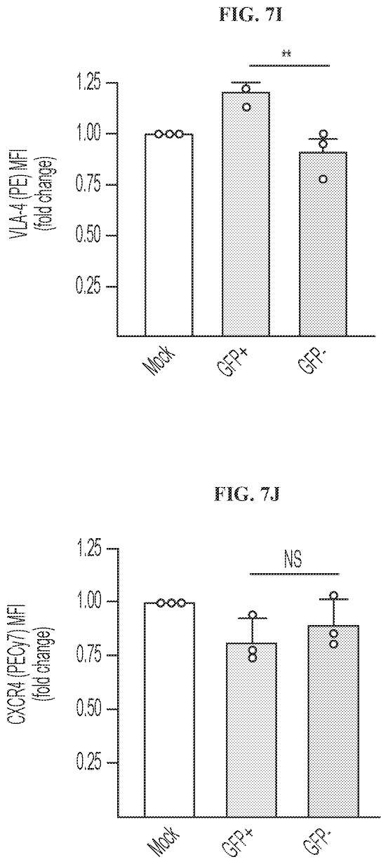

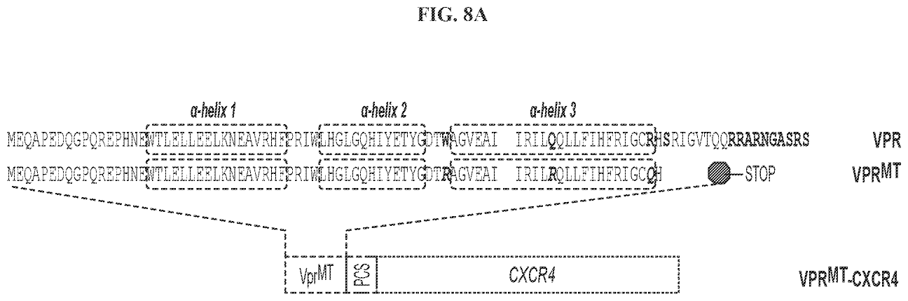

Expression of both VLA-4 and CXCR4 on CD34+HSPC is upregulated by stem cell factor and Flt-3 ligand (Aiuti et al., Eur J Immunol, 1999; Bellucci et al., Bone Marrow Transplant, 1999; Dutt et al., J Imunnol, 1998; Fukuda et al., Blood, 2005; Kovach et al., Blood, 1995; Levesque et al., J Exp Med, 1995; Odemis et al., J Biol Chem, 2002; Peled et al., Science, 1999), cytokines that are used in ex vivo cultures for genetic manipulation, and indeed, both VLA-4 and CXCR4 expression on CD34+HSPC increased with increasing time in culture (FIGS. 7A-7B). Expression of VLA-4 and CXCR4 was highest in CD34+HSPC in G₂M phases and S-phase in contrast to that on the quiescent HSPC. As expected, more CD34+CD38+HPC were cycling, while a great majority of CD34+38−90+HSC were in G0/G1 phase (FIGS. 7C-7D and 7F-7G). To determine if CXCR4 expression was indeed cell cycle dependent, CXCR4 and VLA-4 expression levels on HSC and HPC that were in G0 phase of cell cycle were examined and HPC and HSC in G0 phase had similar CXCR4 and VLA-4 expression (FIGS. 7E and 7H).

However, while lentiviral transduction significantly increased VLA-4 expression on the GM HSPC (GFP+) cells, transduction of HSPC did not have an effect on surface expression of CXCR4 (FIGS. 7I-7J), much like the engraftment data of transduced and untransduced HSPC (FIGS. 1A-1D).

### (D) Differential CXCR4 Expression Provided a Homing Advantage to HPC

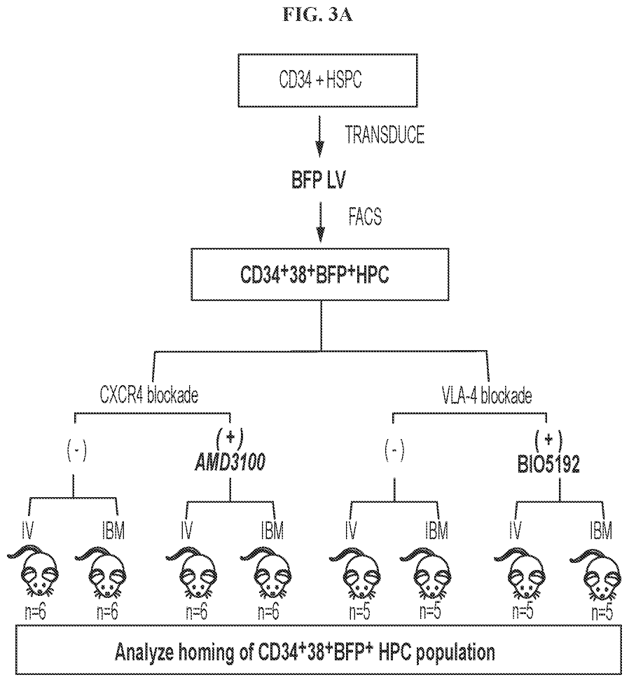

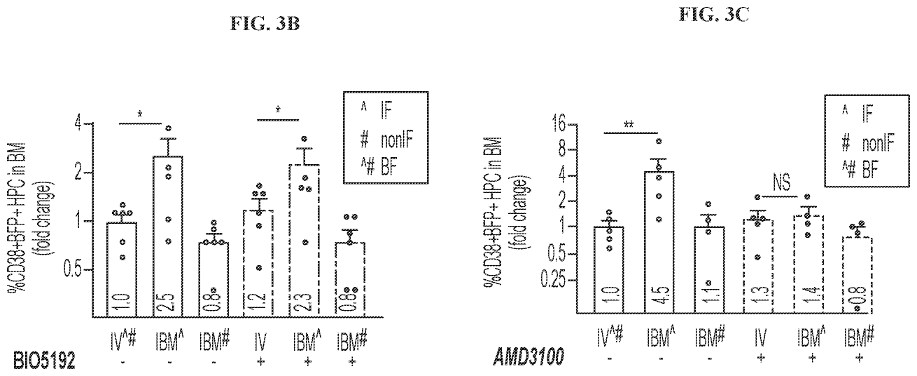

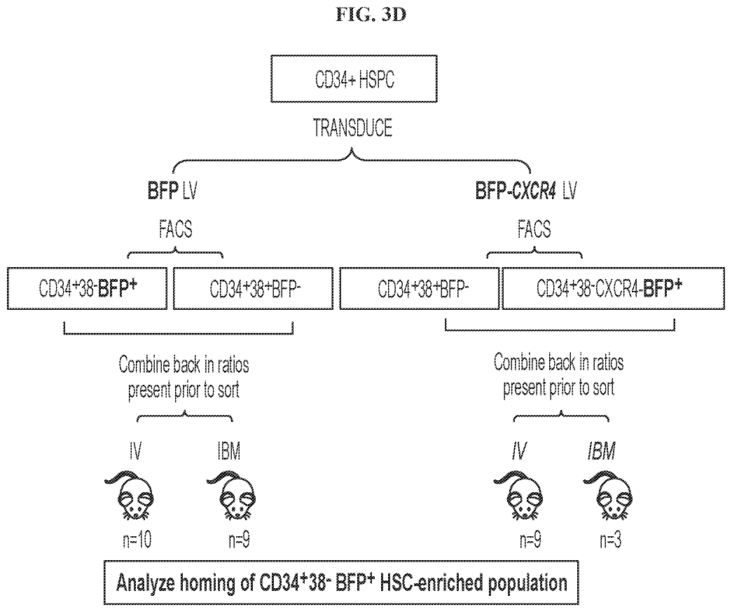

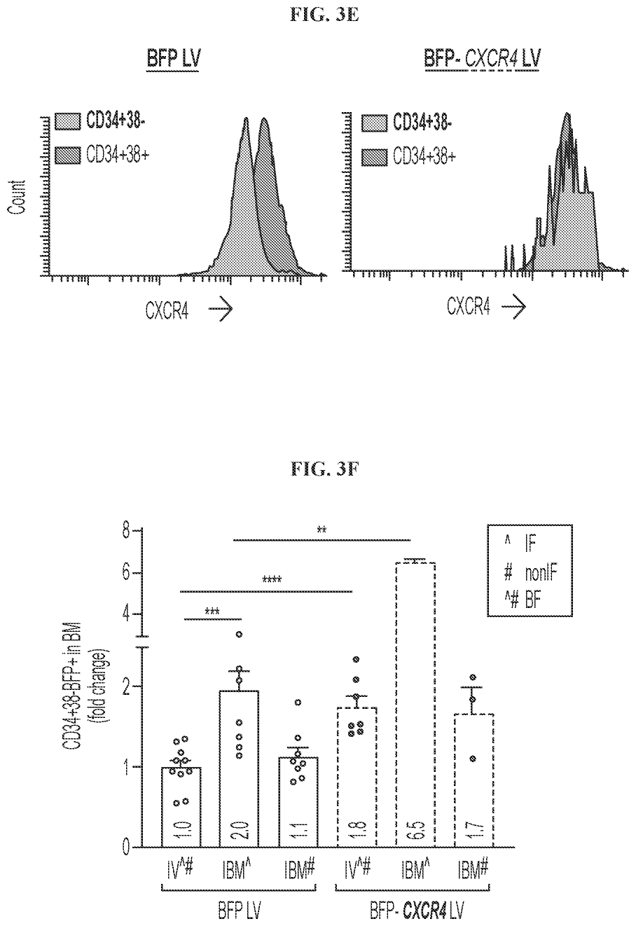

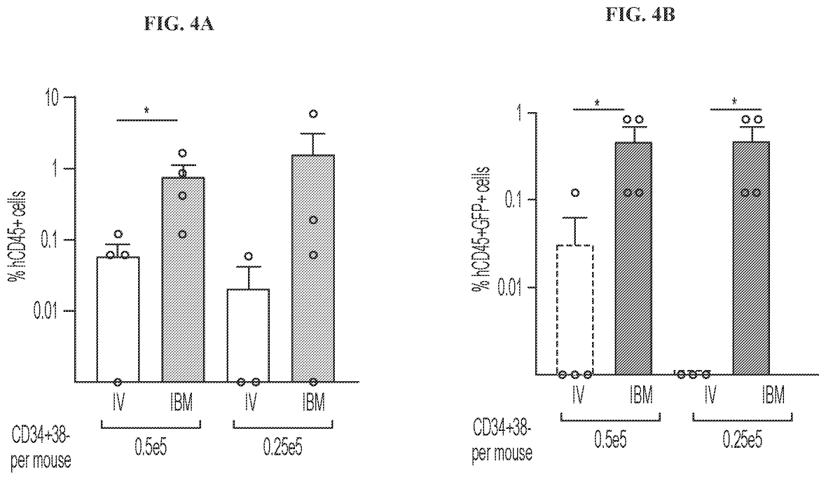

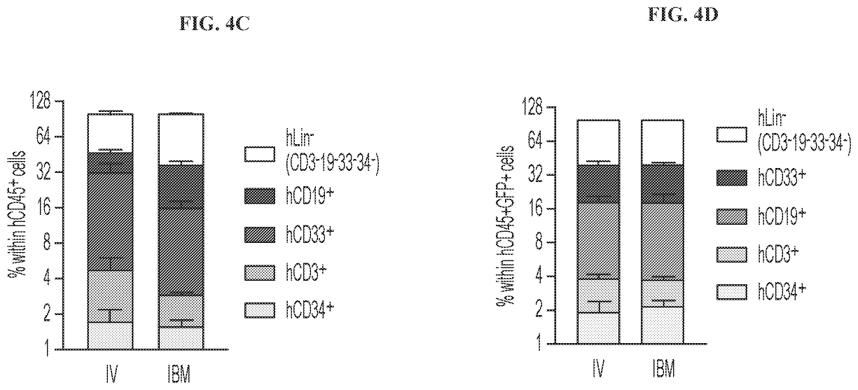

Since HPC have much higher expression of both CXCR4 and VLA-4 compared to HSC, it was determined whether both or one of them promoted HPC retention and homing with direct IBM transplantation. CD34+cells were transduced with a blue fluorescent protein (BFP) encoding LV vector and the CD34+CD38+BFP+HPC were sorted by flow cytometry. VLA-4 and CXCR4 were blocked by exposing the sorted cells to BIO5192 and AMD3100 respectively before injection into irradiated NSG mice (FIG. 3A) to determine their role in retention/homing of CD34+38+HPC into the bone marrow by IBM versus IV administration. In the homing model, control CD34+38+HPC, which were not subjected to CXCR4 or VLA-4 blockade, homed to the bone marrow at significantly higher numbers in the IF with IBM delivery than in the nonIF, and than that with IV delivery (FIGS. 3B-3C), similar to the results seen with homing of CD34+HSPC (FIG. 2A). Blockade of VLA-4 did not alter the CD34+38+HPC homing pattern, as compared to the corresponding controls (FIG. 4B), indicating that VLA-4 homing receptor was not the major contributor to the higher retention/homing of HPC into the injected bone marrow. However, blockade of CXCR4 with AMD3100 significantly reduced human HPC content in the IF to homing levels seen with IV delivery or homing levels in the nonIF (FIG. 3C). Notably, homing of IV injected cells was note blocked with AMD3100, suggesting other homing receptors may play a role. Taken together, these data showed that higher expression of CXCR4, not VLA-4 on HPC, confers a competitive homing advantage.

An alternative explanation for the competitive homing and engraftment advantage of HPC could be that they comprise the vast majority of CD34+HSPC (98-99%), while HSC are a small minority. It was experimentally conferred the HSC-enriched CD34+CD38− population with slightly higher CXCR4 expression than the CD34+CD38+HPC and assessed homing (FIG. 3D). The CD34+HSPC were transduced with a BFP-CXCR4-encoding LV vector or a BFP-encoding LV control vector and CD34+CD38−BFP+HSC-enriched cells were sorted along with untransduced CD34+CD38+BFP−HPC. CXCR4 expression on these populations was assessed before transplant. In control cells, CXCR4 expression on the more primitive CD34+CD38−cells was much lower (MFI 16,930) than on CD34+CD38+HPC (MFI 35,098) (FIG. 3E), consistent with prior data (see, e.g., FIG. 2D). In contrast, CXCR4 expression on the HSC-enriched CD34+CD38−BFP+cells transduced with the BFP-CXCR4 LV vector was now higher (MFI 37,453) than the CD34+CD38+HPC (FIG. 3E). The sorted BFP-HPC population was then mixed with the BFP+CD34+CD38−CXCR4^{high} HSC-enriched cell population in ratios seen in CD34+HSPC prior to sorting, and transplanted them either IV or IBM, and homing of the CD34+CD38−BFP+CXCR4^{high} HSC-enriched cells was assessed. Control CD34+CD38−BFP+cells were also mixed with CD34+CD38+BFP−HPC and transplanted IV or IBM (FIG. 3D) and their homing into bone marrow was assessed. Even when CD34+CD38−BFP+CXCR4^{high} cells were in the minority, they were able to outcompete the CD34+CD38−BFP−HPC that were in the majority, when injected IBM. Furthermore, even IV administration of CD34+CD38−BFP+CXCR4^{high} cells mixed with a majority of CD34+38+HPC led to higher homing levels of CD34+CD38−cells at levels comparable to IBM delivery. Hence, when the CD34+38−HSC-enriched population was conferred with higher CXCR4 expression than HPC, it had higher homing advantage to the bone marrow following IV transplantation, and a remarkably improved homing advantage following direct IBM transplantation (FIG. 3F).

Taken together, these data suggest that: (1) CXCR4, not VLA-4 plays a major role in the differential retention and homing (and therefore likely engraftment) of HPC when delivered IBM, (2) cells highly enriched in HSC, when experimentally endowed with higher CXCR4 expression, have a homing advantage over HPC, and (3) the sheer abundance of HPC did not give them a competitive homing advantage.

### (E). IBM Delivery of an HSC-Enriched Population Enhanced Long-Term Repopulation Compared to IV Delivery

Based on these results, it was hypothesized that removal of the high CXCR4 expressing CD34+CD38+HPC followed by transduction and transplantation of CD34+38−HSC-enriched cell population would confer IBM-delivered HSC a competitive advantage to long-term engraftment in the bone marrow niche. CD38+HPC were magnetically depleted from the CD34+HSPC population and the CD34+CD38−HSC-enriched population was transplanted in NSG mice IV and IBM. 92% purity in CD34+CD38−cells was achieved using the CD34+CD38−Isolation Kit (Miltenyi). Mice were analyzed for long-term engraftment at 24 weeks post-transplant. IBM administration led to significantly increased long-term multi-lineage engraftment compared to IV administration (FIGS. 4A-4B), suggesting competition between HPC and HSC with IBM delivery was removed with HPC depletion. In fact, this advantage was significant in the GM cells where cell doses were limited (FIG. 4B). Lineage analysis of the human graft showed that the human cells (total and GM) were multi-lineage (FIGS. 4C-4D).

### (F) Transient High CXCR4 Expression Conferred a Homing and Engraftment Advantage to CD34+CD38−Long-Term Repopulating Cells

CXCR4 was then increased transiently on GM LTRC to give them a competitive homing and engraftment advantage over HPC. Several treatments can be added to culture to achieve higher CXCR4 expression, specifically CD26 inhibition, mild hyperthermia, prostaglandin E2 (PGE2), glucocorticoid treatment, and HDAC inhibition. See, e.g., Broxmeyer et al., Blood Cells Mol Dis, 2014; Capitano et al., Stem Cells, 2015; Christopherson et al., Science, 2004; Goichberg et al., Blood, 2006; Guo et al., Nat Med, 2017; Hoggatt et al., Blood, 2009; Huang et al., Nat Commun, 2018. However, they increase CXCR4 expression on all HSPC (GM and non-GM HSPC) in culture. Increasing CXCR4 expression only on GM HSC is desirable but poses challenges. CXCR4 cannot be carried on an integrating vector (as was done in the results shown in FIGS. 3D-3F and by Brenner et al., Stem Cells, 2004), because the long-term clinical consequences of this approach are questionable. Specifically, permanent upregulation of CXCR4 leads to myelokathexis and a primary immunodeficiency caused by a hyper functional CXCR4 receptor (Kawai et al., Curr Opin Hemtol, 2009). Regulated expression by placing CXCR4 as an inducible cassette within the transgene vector was not feasible due to low vector titer resulting from large insert size.

To develop an approach that would be clinically translatable, transient high CXCR4 expression was conferred to only GM CD34+CD38−HSC-enriched cells. This HSC-enriched population was chosen instead of HSC because magnetic sorting for this cell population is now commercially available, efforts to make it clinically scalable are underway, and CD34+38−cells have been shown to contain HSC with long-term repopulating ability (Zonari et al., Stem Cell Rep, 2017). The strategy was to deliver CXCR4 protein transiently within the LV vector particle so that GM HSPC obtain the homing advantage and therefore, have higher engraftment than non-GM HSPC.

Viral protein R (Vpr) is a well-characterized small HIV-1 accessory protein that is unique in being present in HIV-1 virions bound to the capsid protein, Gag, via residues in its central region that folds into 3 α-helices (FIG. 8A). These are flanked by unstructured N and C-terminal domains. Vpr mediates early T cell toxicity upon viral cell entry and is therefore excluded from the LV vector packaging plasmids. It mediates its toxicity via nuclear localization domains and its specific residues that cause cell cycle arrest, apoptosis, and interact in the DNA damage response have been well characterized and highlighted in FIG. 8A (Barnitz et al., PLoS One, 2011; Greenwood et al., Cell Rep, 2019; Guenzel et al., Front Microbiol, 2014; Link et al., Nucleic Acids Res, 2006; Marzio et al., J Virol, 1995; Wallet et al., Biochem Pharmacol, 2020; Zhou et al., J Virol, 2000). The C-terminal domain imparts protein stability and has six arginine residues between positions 73 and 96 that potentiate nuclear localization, G₂M arrest and apoptosis. S79 phosphorylation is important for cell cycle arrest. Therefore, VPR was truncated at the 78aa and VPR^{R77Q} mutation was made in the third helix. The third helix forms a leucine-zipper like motif that interacts with DNA damage response (DDR) proteins and UNG2 via W54, and the ubiquitin-proteasome complex via DCAF via Q65 and induces DDR signaling. VPR^{W54R} and VPR^{Q65R} mutations were also made to abrogate binding to UNG2 and DCAF, preventing DDR, and eliminate all changes in the proteome normally triggered by VPR upon viral entry, respectively.

A mutated (W54R, Q65R, and R77Q) and truncated (78 amino acid) version of Vpr, Vpr^{MT}, which retains its folding, oligomerization and gag/capsid binding domains but is devoid of residues and the carboxyl terminal region that mediate cytotoxicity was designed. Next, Vpr^{MT} was fused to CXCR4 cDNA via the HIV-1 protease cleavage site (PCS) to generate Vpr^{MT}-CXCR4. The PCS is recognized by the HIV protease, present in LV vectors along with the two other enzymes, reverse transcriptase and integrase produced by the POL gene during packaging (FIG. 8B). Vpr^{MT} was fused to CXCR4 cDNA using a protease cleavage site of lentiviruses so that CXCR4 was released from Vpr^{MT} upon cell entry and could, therefore, express on the cell surface.

Currently, standard LV vectors are generated by transfecting 293T cells with (a) the packaging plasmids that provide the proteins that form the viral capsid, polymerases (reverse transcriptase, integrase, and protease) and envelope proteins to form the viral particle and (b) the vector genome plasmid that encodes the transgene mRNA. Only the vector genome plasmid has the encapsidation signal allowing packaging of its genetic material/RNA into the vector particles. Vector particles are assembled from the proteins encoded by the packaging plasmids, and no genetic material from the packaging plasmids is encapsidated into viral particles (FIG. 8B).

Adding the Vpr^{MT}-CXCR4 as an additional packaging plasmid would result in packaging this fusion protein within the LV vector particle bound to the capsid protein (FIG. 8D), so that when the vector transduces cells, the fusion protein would be released in transduced cells, and cleaved by the protease at the PCS, releasing CXCR4 to come to the cell surface. This strategy allowed delivery of CXCR4 as a protein only on cells transduced by the LV vector, while the vector delivered and integrated the therapeutic transgene into the cellular genome.

Empty LV^{CXCR} particles, or vector-like particles (VLP), that were generated without co-transfecting the vector transgene plasmid, were generated using the Vpr^{MT}-CXCR4 plasmid in addition to the standard packaging plasmids (Gag-Pol, Rev, and VSV-G) (FIG. 8C) to determine if this strategy results in cell surface expression of CXCR4 in the cells transduced with LV^{CXCR4} vector-like particles that contain no genetic material. K562 cells, which normally do not express CXCR4, were transduced at increasing concentrations of LV^{CXCR4} VLP. CXCR4 surface expression on the transduced K562 cells increased in a VLP-dose-dependent manner, demonstrating that Vpr^{MT}-CXCR4 protein, delivered attached to the lentivirus vector capsid protein from the LV^{CXCR4} vector, indeed resulted in cell surface expression of CXCR4.

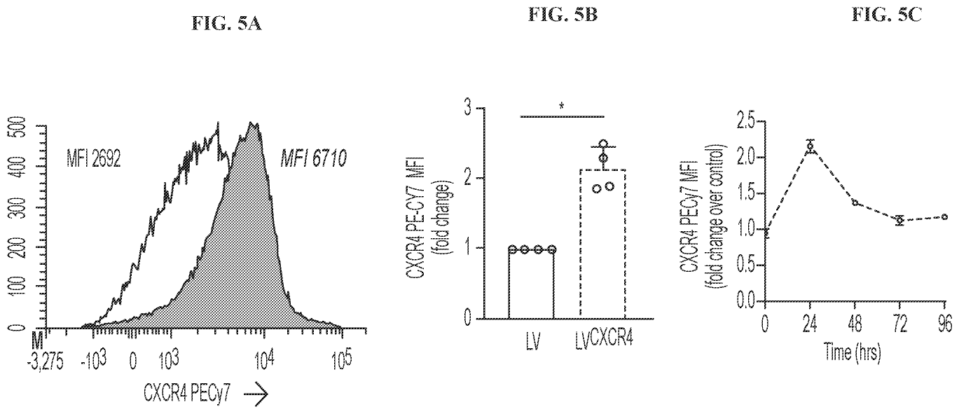

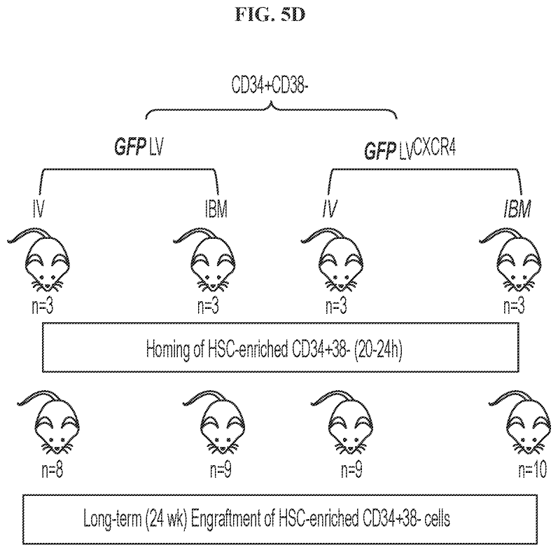

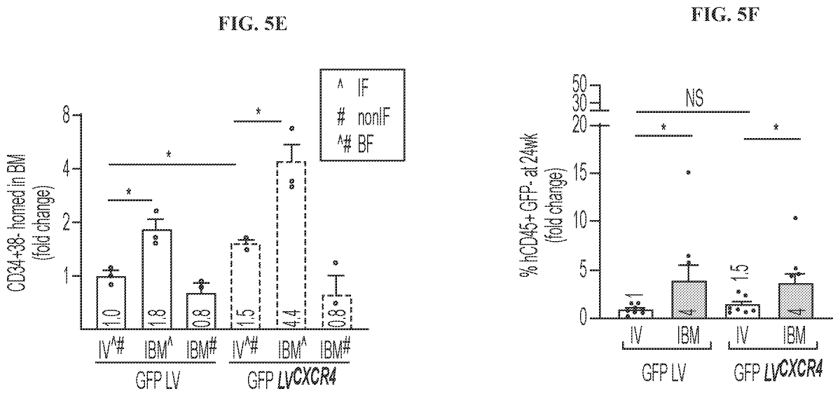

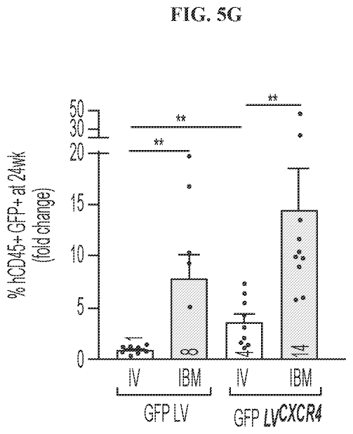

Next, a GFP vector either packaged with Vpr^{MT}-CXCR4 and other standard LV packaging plasmids (GFP LV^{CXCR4} vector) or packaged using only the standard LV packaging plasmids (GFP LV vector) was generated (FIG. 8D) and both vectors were tested in MPB CD34+HSPC. In primary MPB CD34+HSPC, transduction with GFP LV^{CXCR4} vector was sufficient to induce a significantly higher CXCR4 expression (a 2-fold higher CXCR4 MFI) on CD34+HSPC as compared to CD34+HSPC transduced with the control GFP LV vector (FIGS. 5A-5B). Furthermore, since the CXCR4 was delivered as a protein, the expression of CXCR4 in the GFP LV^{CXCR4}-transduced CD34+HSPC was transient, peaking at 24 hours and returning to baseline levels by 72 hours post-transduction (FIG. 5C). Notably, in the Vpr^{MT}-CXCR4 fusion construct, Vpr domains/residues known to cause cytotoxicity to T cells were removed, while retaining portions that allow proper protein folding and capsid-binding.

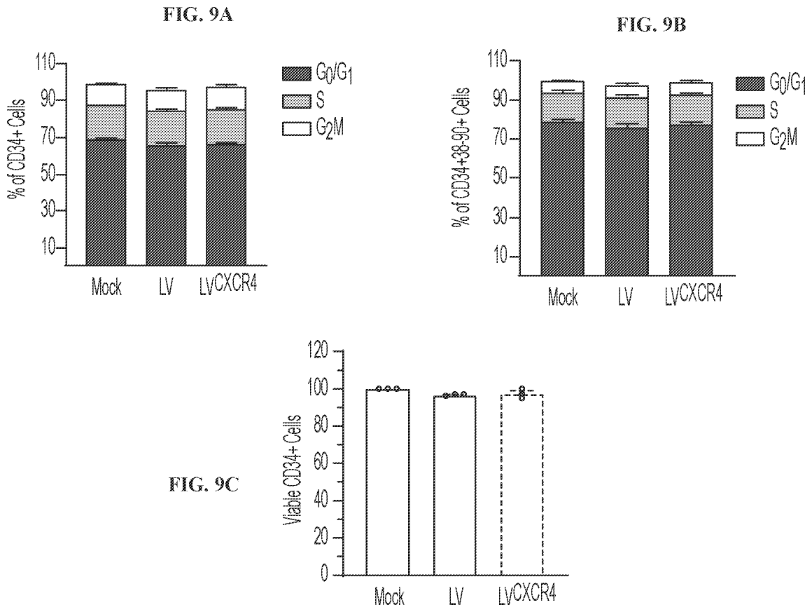

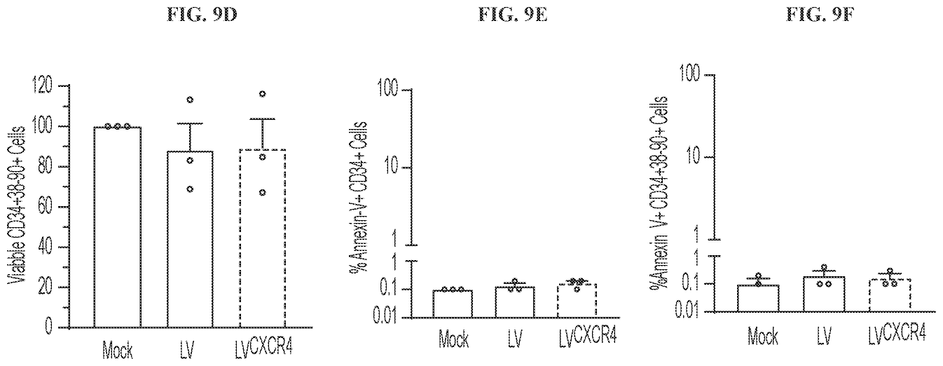

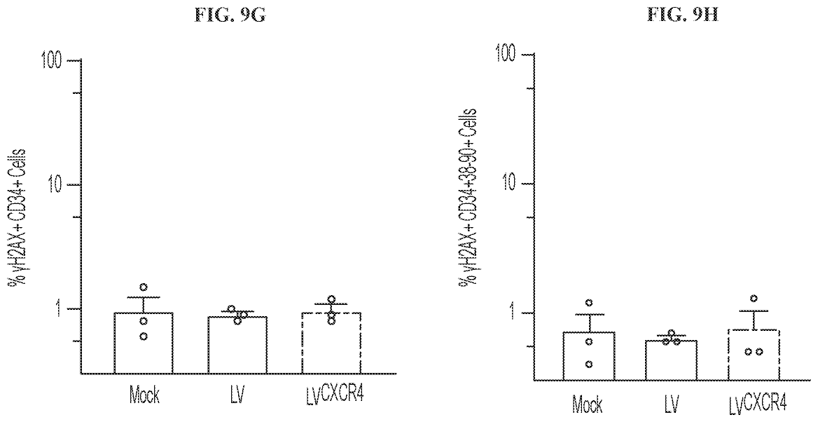

In order to ensure that this fusion construct delivered via LV^{CXCR4} was not associated with increased cytotoxicity to HSPC compared to a standard control LV vector, MPB CD34+cells were transduced with GFP LV^{CXCR4} and GFP LV and assessed for the known toxicities of Vpr: G₂M cell cycle arrest, cell viability, apoptosis and induction of DNA damage response in CD34+HSPC and CD34+CD38−CD90+HSC were assessed (FIGS. 9A-9H). The Vpr^{MT}-CXCR4 delivery via the LV^{CXCR4} showed no toxicity as compared to mock-transduced and standard LV-transduced HSPC and HSC. Hence, this truncated and mutated Vpr^{MT}-CXCR4 delivered CXCR4 to the surface of transduced cells, expressed transiently, without inducing cytotoxicity.

CD34+CD38−HSC-enriched cells were then transduced with the GFP LV^{CXCR}4 vector or a control GFP LV vector and transplanted via IV or IBM in NSG mice to assess homing and engraftment (FIG. 5D). For assessment of homing, CD34+CD38−HSC-enriched cells were labeled with PKH26 before IV or IBM delivery into irradiated NSG mice to allow accurate detection because GFP expression had not yet peaked (it takes 36-60 hours for peak transgene expression following lentiviral post-entry, reverse transcription and integration into the cellular genome, followed by transcription and protein expression of GFP). Homing to the bone marrow was assessed at 20 hours post-delivery (FIG. 5E). There was a significant increase in CD34+CD38−HSC-enriched cell homing to the bone marrow after IV delivery of GFP LV^{CXCR4} vector transduced CD34+CD38−cells as compared to the IV delivery of control GFP LV vector transduced CD34+CD38−cells. In fact, homing of GFP LV^{CXCR4} transduced CD34+CD38−cells delivered IV was at levels of homing comparable to those in the IF of the IBM GFP LV control mouse group (FIG. 5E). Furthermore, within the IBM-injected mice, homing of GFP LV^{CXCR4} transduced CD34+CD38−cells to the bone marrow was more than twice the levels seen in the GFP LV transduced CD34+CD38−HSC-enriched controls in the IF, and more than four times that of IV delivery (FIG. 5E). These data indicated that transient upregulation of CXCR4 conferred a strong homing advantage to CD34+CD38−HSC-enriched cells when transplanted IV, and that this homing advantage was even stronger when transplanted directly into bone marrow. The fact that this method of CXCR4 protein delivery utilized vector packaging and not the vector genome means that any vector genome/transgene could be packaged using this additional fusion plasmid, to provide a 2-4-fold homing advantage, specifically to GM cells, which in turn may confer higher engraftment to GM HSC.

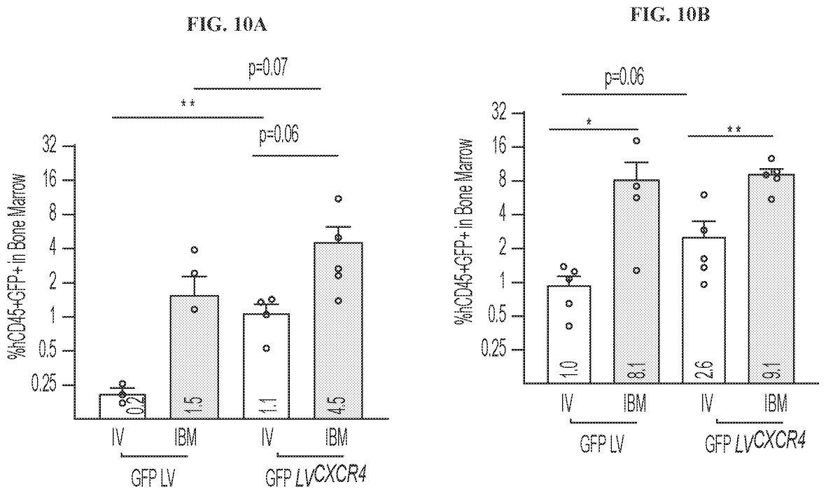

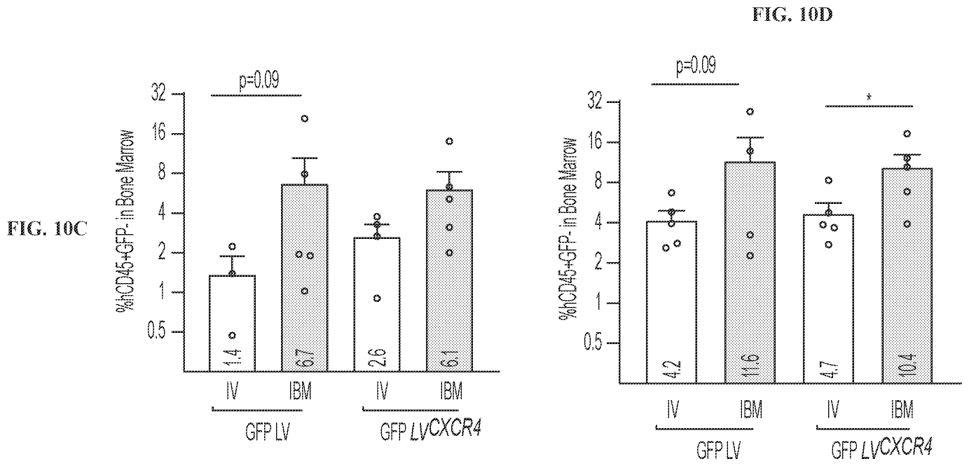

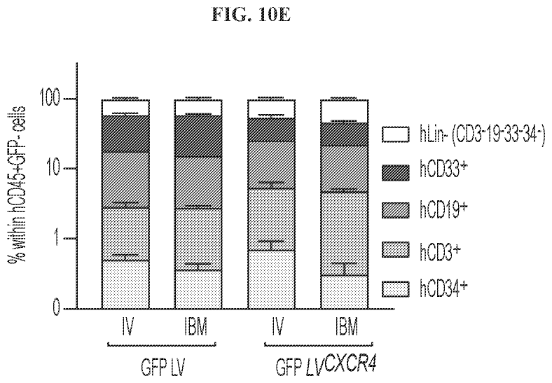

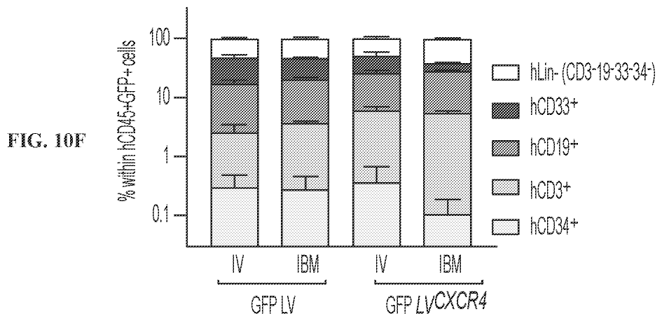

Next, CD34+CD38−HSC-enriched cells were sorted, transduced with GFP LV^{CXCR4} vector or GFP LV control vector, and transplanted them IV or IBM in two limiting dilutions to irradiated NSG mice to assess engraftment of the long-term repopulating HSC at 6 months post-transplant (FIG. 5F-5G). The engraftment of GFP LV^{CXCR4} GM CD34+CD38−cells transplanted IV engrafted significantly (4-fold) higher than GFP LV GM CD34+CD38−cells transplanted IV (FIG. 5F and FIGS. 10A-10D). Following IBM delivery, GFP LV^{CXCR4} transduced CD34+CD38−HSC-enriched cells had a nearly 2-fold higher engraftment than IBM delivery of GFP LV transduced CD34+CD38−cells. There was no significant difference in long-term repopulation of untransduced cells between GFP LV and GFP LV^{CXCR4} groups, confirming that the engraftment advantage seen in transduced cells was due to the transient increase in CXCR4 expression (FIG. 5G and FIGS. 10A-10D). The slightly higher engraftment of untransduced IV transplanted cells (FIGS. 5F and 10C) was likely because LV preparations often contain some defective viral particles, which, like VLP, would deliver CXCR4 protein but not result in an effective transduction (transgene transfer or GFP+cells). Hence, some untransduced cells may have received defective particles, which would still carry the CXCR4 protein. Cells transduced with both GFP LV and GFP LV^{CXCR4} produced full multilineage repopulation at 24 weeks (FIGS. 10E-10F), confirming engraftment of HSC. Taken together, LV vectors used to transduce HSC-enriched cells could be used to confer a homing and engraftment advantage specifically to the transduced HSC-enriched populations. With limiting GM HSC dose, IBM delivery using this method could result in significantly higher long-term engraftment.

Improving the engraftment of GM adult HSC to achieve high levels of engraftment with a limited cell dose is essential to broadening the use of gene therapy. Herein, using a human NSG xenograft model, direct bone marrow transplantation of GM HSPC was shown to enhance HPC engraftment but did not significantly improve engraftment of LTRC, and the underlying mechanism was shown. The mechanism was then utilized by demonstrating transient CXCR4 upregulation in a clinically translatable LTRC population to specifically increase GM LTRC engraftment when transplanted IV and remarkably enhance engraftment of GM LTRC when transplanted IBM.

In sum, this study identified the mechanism that explains disparate short-and long-term engraftment data reported on IBM transplantation. While CXCR4 is a well-established homing receptor on HSPC, higher CXCR4 expression on HPC gave HPC a homing and engraftment advantage over lower CXCR4-expressing HSC following IBM delivery. Removing CXCR4^{high} HPC and transplanting an HSC-enriched population removed competition and resulted in improved long-term engraftment following direct bone marrow transplantation. In an effort to minimize LV vector usage and reduce associated costs, transplantation of an HSC-enriched CD34+CD38− or CD34+CD38−CD90+ population is coming into favor (Zonari et al., Stem Cell Rep, 2017, Masiuk et al., Mol Ther, 2017). As this becomes the standard of care, direct bone marrow transplantation may be a relevant clinical option to reduce homing losses and achieve improved transplant success.

Alternatively, transient protein delivery of CXCR4 in the LV particle was shown to be useful to improve homing and engraftment of GM HSPC specifically, even when delivered IV, without altering the homing and engraftment of untransduced HSPC. All cytotoxic domains can be removed from Vpr while retaining gag-binding. Using protein delivery allows for the cells to have an advantage during the critical homing time period but does not risk causing future immunodeficiency resulting from permanent CXCR4 upregulation. Additionally, by fusing CXCR4 to Vpr and carrying it as a protein rather than as a LV gene, the issues of transgene size and double transduction that have impaired previous efforts were avoided.

CXCR4 delivered via VLP was shown to increase CXCR4 cell surface expression, and hence VLP^{CXCR4} could be used in conjunction with gene editing approaches and even UCB transplants, expanding the use of this approach beyond LV vectors. Finally, by either using CXCR4 or substituting CXCR4 with other homing molecules, this Vpr protein delivery method could be used to target other cell-based therapies such as chimeric antigen receptor (CAR)-T and CAR-NK cells to their specific homing sites.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A virus-like particle (VLP), comprising a protein shell that comprises (a) a protein of interest, which optionally is a cell homing molecule, and (b) one or more viral surface proteins; wherein when entering a host cell, the protein of interest detaches from the VLP and delivers to the host cell; optionally wherein the protein of interest is displayed on the surface of the host cell.

2. The VLP of claim 1, further comprising a nucleic acid, which is encapsulated by the protein shell, wherein the nucleic acid comprises a transgene of interest; and wherein when entering the host cell, the VLP delivers to the host cell both the cell homing molecule and the transgene.

3. The VLP of claim 1 or claim 2, wherein the protein of interest is the cell homing molecule, which is a fusion protein comprising a CXCR4 polypeptide fused to a viral protein R (Vpr) fragment, and wherein the Vpr fragment is devoid of toxicity.

4. The VLP of claim 3, wherein the Vpr fragment comprises a truncation of a carboxyl terminal region and/or one or more mutations at positions W54, Q65, and R77 as compared with the wild-type counterpart.

5. The VLP of claim 4, wherein the one or more mutations are amino acid substitutions of W54R, Q65R, and R77Q.

6. The VLP of claim 4 or claim 5, wherein the Vpr fragment comprises an amino acid sequence at least 80% identical to residues 1-78 of SEQ ID NO:1 and comprises one or more of the mutations.

7. The VLP of claim 6, wherein the Vpr comprises the amino acid sequence of SEQ ID NO:2.

8. The VLP of any one of claims 3-7, wherein the fusion protein further comprises a protease cleavage peptide located between the CXCR4 polypeptide and the Vpr fragment; and wherein cleavage at the protease cleavage peptide releases the CXCR4 polypeptide.

9. The VLP of claim 8, wherein the VLP further comprises a protease that recognize the protease cleavage peptide.

10. The VLP of any one of claims 1-9, wherein the VLP is a lentivirus-like particle.

11. The VLP of claim 10, wherein the protease cleavage peptide comprises a polybasic cleavage site (PCS).

12. A method for delivering a protein of interest and optionally a transgene to host cells, the method comprising:
contacting a virus-like particle (VLP) set forth in any one of claims 1-11 to a population of host cells to allow entry of the VLP into the host cells, thereby delivering to the host cells the cell homing molecule and optionally the transgene contained in the VLP.

13. The method of claim 12, wherein the population of host cells comprise hematopoietic cells.

14. The method of claim 13, wherein the hematopoietic cells are umbilical cord blood (UCB) cells.

15. The method of claim 13 or claim 14, wherein the hematopoietic cells comprise immune cells, which optionally are T cells and/or NK cells.

16. The method of claim 15, wherein the transgene encodes a chimeric antigen receptor.

17. The method of claim 13, wherein the hematopoietic cells are hematopoietic progenitor cells (HPCs), hematopoietic stem cells (HSCs), or a combination thereof.

18. The method of claim 13, wherein the hematopoietic cells comprise CD34+cells.

19. The method of claim 13, wherein the hematopoietic cells comprise CD34⁺/CD38⁺cells.

20. The method of claim 13, wherein the hematopoietic cells comprise CD34⁺/CD38⁻cells.

21. The method of claim 13, wherein the hematopoietic cells comprise CD34⁺/CD38⁻/CD90⁺cells.

22. A fusion protein comprising a CXCR4 polypeptide fused to a viral protein R (Vpr) fragment, wherein the Vpr fragment is devoid of toxicity.

23. The fusion protein of claim 22, wherein the Vpr fragment comprises a truncation of a carboxyl terminal region and/or one or more mutations at positions W54, Q65, and R77 as compared with the wild-type counterpart.

24. The fusion protein of claim 23, wherein the one or more mutations are amino acid substitutions of W54R, Q65R, and R77Q.

25. The fusion protein of claim 23 or claim 24, wherein the Vpr fragment comprises an amino acid sequence at least 80% identical to 1-78 amino acid residues of SEQ ID NO:1 and comprises one or more of the mutations.

26. The fusion protein of claim 25, wherein the Vpr comprises the amino acid sequence of SEQ ID NO:2.

27. The fusion protein of any one of claims 22-26, wherein the fusion protein further comprises a protease cleavage peptide located between the CXCR4 polypeptide and the Vpr fragment; and wherein cleavage at the protease cleavage peptide releases the CXCR4 polypeptide.

28. The fusion protein of claim 27, wherein the protease cleavage peptide comprises a polybasic cleavage site (PCS).

29. A nucleic acid, comprising a nucleotide sequence encoding a fusion protein set forth in any one of claims 22-28.

30. The nucleic acid of claim 29, which is an expression vector, wherein the nucleotide sequence encoding the fusion protein is in operable linkage to a promoter.

31. A host cell comprising the nucleic acid of claim 29 or claim 30.

32. A population of hematopoietic cells comprising modified hematopoietic cells, which comprises an exogenous cell homing molecule and optionally a transgene of interest; wherein the modified hematopoietic cells have higher homing and engraftment activity relative to counterpart hematopoietic cells lacking the exogenous cell homing molecule post transplantation to a subject.

33. The population of hematopoietic cells of claim 32, wherein the cell homing molecule is a CXCR4 protein, which optionally is set forth in any one of claims 22-28.

34. The population of hematopoietic cells of claim 33, wherein the modified hematopoietic cells comprise:
(a) CD34⁺cells;
(b) CD34⁺/CD38⁺cells;
(c) CD34⁺/CD38⁻cells, or
(d) CD34⁺/CD38⁻/CD90⁺cells.

35. The population of hematopoietic cells of claim 33, wherein the modified hematopoietic cells comprise the following characteristics: (a) CD34⁺ and (b) one or more of CD38⁻, CD90⁺, CD45RA⁻, CD49F⁺, and CD133⁺.

36. The population of hematopoietic cells of claim 32, which are produced by a method set forth in any one of claims 12-21.

37. A method for delivering hematopoietic cells to a subject, the method comprising: administering a population of hematopoietic cells set forth in any one of claims 32-36 to a subject in need thereof,
wherein the hematopoietic cells have enhanced homing and/or engraftment capacity as compared with counterpart hematopoietic cells lacking the exogenous cell homing molecule.

38. The method of claim 37, wherein the subject is a human patient in need of stem cell transplantation and/or gene therapy.

39. The method of claim 37 or claim 38, wherein the population of hematopoietic cells are administered to the subject by intravenous injection or intra-bone marrow (IBM) injection.

40. The method of any one of claims 37-39, wherein the population of hematopoietic cells is autologous to the subject.

41. A gene therapy method, comprising:
(i) isolating a population of hematopoietic cells from a first subject;
(ii) introducing into the population of hematopoietic cells (a) a transgene encoding a therapeutic agent and (b) a cell homing molecule to produce a population of modified hematopoietic cells; and
(iii) administering the modified hematopoietic cells to a second subject.

42. The gene therapy method of claim 41, wherein the first subject and the second subject are an identical human patient.

43. The gene therapy method of claim 41 or claim 42, wherein in step (ii), the transgene and the cell homing molecule are introduced into the population of hematopoietic cells concurrently.

44. The gene therapy method of any one of claims 41-43, wherein the cell homing molecule is a CXCR4 protein set forth in any one of claims 22-28.

45. The gene therapy method of any one of claims 41-44, wherein the population of hematopoietic cells isolated in step (i) comprises:
(a) CD34⁺cells;
(b) CD34⁺/CD38⁺cells;
(c) CD34⁺/CD38⁻cells, or
(d) CD34⁺/CD38⁻/CD90⁺cells.

46. The gene therapy method of any one of claims 41-45, wherein the population of hematopoietic cells comprise the following characteristics: (a) CD34⁺ and (b) one or more of CD38⁻, CD90⁺, CD45RA⁻, CD49F⁺, and CD133⁺.

47. The gene therapy method of any one of claims 41-46, wherein the modified hematopoietic cells are administered to the second subject by intravenous infusion or intra-bone marrow injection.

48. A method for improving engraftment of long-term repopulating cells (LTRCs) in a subject, the method comprising administering a population of hematopoietic cells comprising LTRCs to a subject in need thereof by intra-bone marrow (IBM) injection, wherein the LTRCs comprise CD34+ and CD38−cells, and wherein the population of hematopoietic cells comprise no more than 10-20%% CD34+and CD38+cells.

49. The method of claim 48, wherein the LTRCs comprise CD34⁺/CD38⁻/CD90⁺cells.

50. The method of claim 49, wherein the LTRCs comprise CD90⁻ and/or CD45RA⁻cells.

51. The method of any one of claims 48-50, wherein the subject is a human patient in need of stem cell transplantation and/or gene therapy.

52. The method of any one of claims 48-51, wherein the LTRCs are genetically engineered to carry a transgene of interest.

53. The method of any one of claims 48-52, wherein the LTRCs are autologous to the subject.

54. The method of any one of claims 48-52, wherein the LTRCs are allogeneic to the subject.
